(19) United States
(12) Patent Application Publication
(10) Pub. No.: US 2021/0267973 A1
(43) Pub. Date: Sep. 2, 2021

(54) COMBINATION OF A TYPE II PROTEIN ARGININE METHYLTRANSFERASE INHIBITOR AND AN ICOS BINDING PROTEIN TO TREAT CANCER

(71) Applicant: GLAXOSMITHKLINE INTELLECTUAL PROPERTY DEVELOPMENT LIMITED, Brentford, Middlesex (GB)

(72) Inventors: Olena I. BARBASH, Collegeville, PA (US); Andrew Mark FEDORIW, Collegeville, PA (US); Susan KORENCHUK, Collegeville, PA (US); Christian S. SHERK, Collegeville, PA (US)

(21) Appl. No.: 17/052,606

(22) PCT Filed: May 24, 2019
(86) PCT No.: PCT/IB2019/054346

§ 371 Date: Nov. 3, 2020

Related U.S. Application Data
(60) Provisional application No. 62/678,398, filed on May 31, 2018.

(51) Int. Cl. A61K 31/506; A61K 39/395; C07K 16/28; A61K 9/00; A61P 35/00

(52) U.S. Cl. CPC A61K 31/506; A61K 39/39533; C07K 16/2818; A61K 9/0053; A61K 9/0019; A61K 2039/505; C07K 2317/75; C07K 2317/565; C07K 2317/56; C07K 2317/33; A61P 35/00

(57) ABSTRACT

In one aspect, the present invention provides a method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding portion thereof. In another aspect, the present invention provides a Type II protein arginine methyltransferase (Type II PRMT) inhibitor and an ICOS binding protein or antigen binding fragment thereof for use in treating cancer in a human in need thereof.

### FIELD OF THE INVENTION

The present invention relates to a method of treating cancer in a mammal and to combinations useful in such treatment. In particular, the present invention relates to combinations of Type II protein arginine methyltransferase (Type II PRMT) inhibitors and immuno-modulatory agents, such as anti-ICOS antibodies.

### BACKGROUND OF THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (reviewed in Yang, Y. & Bedford, M. T. Protein arginine methyltransferases and cancer. Nat Rev Cancer 13, 37-50, doi:10.1038/nrc3409 (2013); Lee, Y. H. & Stallcup, M. R. Minireview: protein arginine methylation of nonhistone proteins in transcriptional regulation. Mol Endocrinol 23, 425-433, doi: 10.1210/me.2008-0380 (2009)). In mammalian cells, methylated arginine exists in three major forms: ω-N^{G}-monomethyl-arginine (MMA), ω-N^{G},N^{G}-asymmetric dimethyl arginine (ADMA), or ω-N^{G},N′^{G}-symmetric dimethyl arginine (SDMA). Each methylation state can affect protein-protein interactions in different ways and therefore has the potential to confer distinct functional consequences for the biological activity of the substrate (Yang, Y. & Bedford, M. T. Protein arginine methyltransferases and cancer. Nat Rev Cancer 13, 37-50, doi:10.1038/nrc3409 (2013)).

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine. This family of proteins is comprised of 10 members of which 9 have been shown to have enzymatic activity (Bedford, M. T. & Clarke, S. G. Protein arginine methylation in mammals: who, what, and why. Mol Cell 33, 1-13, doi:10.1016/j.molcel.2008.12.013 (2009)). The PRMT family is categorized into four sub-types (Type I-IV) depending on the product of the enzymatic reaction. Type IV enzymes methylate the internal guanidino nitrogen and have only been described in yeast (Fisk, J. C. & Read, L. K. Protein arginine methylation in parasitic protozoa. Eukaryot Cell 10, 1013-1022, doi:10.1128/EC.05103-11 (2011)); types I-III enzymes generate monomethyl-arginine (MMA, Rme1) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively. Type II PRMTs include PRMT5, and PRMT9, however, PRMT5 is the primary enzyme responsible for formation of symmetric dimethylation. Type I enzymes include PRMT1, PRMT3, PRMT4, PRMT6 and PRMT8. PRMT1, PRMT3, PRMT4, and PRMT6 are ubiquitously expressed while PRMT8 is largely restricted to the brain (reviewed in Bedford, M. T. & Clarke, S. G. Protein arginine methylation in mammals: who, what, and why. Mol Cell 33, 1-13, doi:10.1016/j.molcel.2008.12.013 (2009)).

PRMT5 functions in several types of complexes in the cytoplasm and the nucleus and binding partners of PRMT5 are required for substrate recognition and selectivity. Methylosome protein 50 (MEP50) is a known cofactor of PRMT5 that is required for PRMT5 binding and activity towards histones and other substrates (Ho M C, et al. Structure of the arginine methyltransferase PRMT5-MEP50 reveals a mechanism for substrate specificity. PLoS One. 2013; 8(2)).

PRMT5 symmetrically methylates arginines in multiple proteins, preferentially in regions rich in arginine and glycine residues (Karkhanis V, et al. Versatility of PRMT5-induced methylation in growth control and development. Trends Biochem Sci. 2011 December; 36(12):633-41). PRMT5 methylates arginines in various cellular proteins including splicing factors, histones, transcription factors, kinases and others (Karkhanis V, et al. Versatility of PRMT5-induced methylation in growth control and development. Trends Biochem Sci. 2011 December; 36(12):633-41). Methylation of multiple components of the spliceosome is a key event in spliceosome assembly and the attenuation of PRMT5 activity through knockdown or gene knockout leads to disruption of cellular splicing (Bezzi M, et al. Regulation of constitutive and alternative splicing by PRMT5 reveals a role for Mdm4 pre-mRNA in sensing defects in the spliceosomal machinery. Genes Dev. 2013 Sep. 1; 27(17):1903-16). PRMT5 also methylates histone arginine residues (H3R8, H2AR3 and H4R3) and these histone marks are associated with transcriptional silencing of tumor suppressor genes, such as RB and ST7 (Wang L, Pal S, Sif S. Protein arginine methyltransferase 5 suppresses the transcription of the RB family of tumor suppressors in leukemia and lymphoma cells. Mol Cell Biol. 2008 October; 28(20):6262-77). Additionally, symmetric dimethylation of H2AR3 has been implicated in the silencing of differentiation genes in embryonic stem cells (Tee W W, Pardo M, Theunissen T W, Yu L, Choudhary J S, Hajkova P, Surani M A. Prmt5 is essential for early mouse development and acts in the cytoplasm to maintain ES cell pluripotency. Genes Dev. 2010 Dec. 15; 24(24):2772-7). PRMT5 also plays a role in cellular signaling, through the methylation of EGFR and PI3K (Hsu J M, Chen C T, Chou C K, Kuo H P, Li L Y, Lin C Y, Lee H J, Wang Y N, Liu M, Liao H W, Shi B, Lai C C, Bedford M T, Tsai C H, Hung M C. Crosstalk between Arg 1175 methylation and Tyr 1173 phosphorylation negatively modulates EGFR-mediated ERK activation. Nat Cell Biol. 2011 February; 13(2):174-81; Wei T Y, Juan C C, Hisa J Y, Su L J, Lee Y C, Chou H Y, Chen J M, Wu Y C, Chiu S C, Hsu C P, Liu K L, Yu C T. Protein arginine methyltransferase 5 is a potential oncoprotein that upregulates G1 cyclins/cyclin-dependent kinases and the phosphoinositide 3-kinase/AKT signaling cascade. Cancer Sci. 2012 September; 103(9):1640-50).

Increasing evidence suggests that PRMT5 is involved in tumorigenesis. PRMT5 protein is overexpressed in a number of cancer types, including lymphoma, glioma, breast and lung cancer and PRMT5 overexpression alone is sufficient to transform normal fibroblasts (Pal S, Baiocchi R A, Byrd J C, Grever M R, Jacob S T, Sif S. Low levels of miR-92b/96 induce PRMT5 translation and H3R8/H4R3 methylation in mantle cell lymphoma. EMBO J. 2007 Aug. 8; 26(15):3558-69; Ibrahim R, et al. Expression of PRMT5 in lung adenocarcinoma and its significance in epithelial-mesenchymal transition. Hum Pathol. 2014 July; 45(7):1397-405; Powers M A, et al. Protein arginine methyltransferase 5 accelerates tumor growth by arginine methylation of the tumor suppressor programmed cell death 4. Cancer Res. 2011 Aug. 15; 71(16):5579-87; Yan F, et al. Genetic validation of the protein arginine methyltransferase PRMT5 as a candidate therapeutic target in glioblastoma. Cancer Res. 2014 Mar. 15; 74(6):1752-65). Knockdown of PRMT5 often leads to a decrease in cell growth and survival in cancer cell lines. In breast cancer, high PRMT5 expression, together with high PDCD4 (programmed cell death 4) levels predict overall poor survival (Powers M A, et al. Cancer Res. 2011 Aug. 15; 71(16):5579-87). PRMT5 methylates PDCD4 altering tumor-related functions. Co-expression of PRMT5 and PDCD4 in an orthotopic model of breast cancer promotes tumor growth. High expression of PRMT5 in glioma is associated with high tumor grade and overall poor survival and PRMT5 knockdown provides a survival benefit in an orthotopic glioblastoma model (Yan F, et al. Genetic validation of the protein arginine methyltransferase PRMT5 as a candidate therapeutic target in glioblastoma. Cancer Res. 2014 Mar. 15; 74(6):1752-65). Increased PRMT5 expression and activity contribute to silencing of several tumor suppressor genes in glioma cell lines.

The strongest mechanistic link currently described between PRMT5 and cancer is in mantle cell lymphoma (MCL). PRMT5 is frequently overexpressed in MCL and is highly expressed in the nuclear compartment where it increases the levels of histone methylation and silences a subset of tumor suppressor genes. Recent studies uncovered the role of miRNAs in the upregulation of PRMT5 expression in MCL. More than 50 miRNAs are predicted to anneal to the 3′ untranslated region of PRMT5 mRNA. It was reported that miR-92b and miR-96 levels inversely correlate with PRMT5 levels in MCL and that the downregulation of these miRNAs in MCL cells results in the upregulation PRMT5 protein levels. Cyclin D1, the oncogene that is translocated in the vast majority of MCL patients, associates with PRMT5 and through a cdk4-dependent mechanism increases PRMT5 activity (Aggarwal P, et al. Nuclear cyclin D1/CDK4 kinase regulates CUL4 expression and triggers neoplastic growth via activation of the PRMT5 methyltransferase. Cancer Cell. 2010 Oct. 19; 18(4):329-40). PRMT5 mediates the suppression of key genes that negatively regulate DNA replication allowing for cyclin D1-dependent neoplastic growth. PRMT5 knockdown inhibits cyclin D1-dependent cell transformation causing death of tumor cells. These data highlight the important role of PRMT5 in MCL and suggest that PRMT5 inhibition could be used as a therapeutic strategy in MCL.

In other tumor types, PRMT5 has been postulated to play a role in differentiation, cell death, cell cycle progression, cell growth and proliferation. While the primary mechanism linking PRMT5 to tumorigenesis is unknown, emerging data suggest that PRMT5 contributes to regulation of gene expression (histone methylation, transcription factor binding, or promoter binding), alteration of splicing, and signal transduction. PRMT5 methylation of the transcription factor E2F1 decreases its ability to suppress cell growth and promote apoptosis (Zheng S, et al. Arginine methylation-dependent reader-writer interplay governs growth control by E2F-1. Mol Cell. 2013 Oct. 10; 52(1):37-51). PRMT5 also methylates p53 (Jansson M, et al. Arginine methylation regulates the p53 response. Nat Cell Biol. 2008 December; 10(12):1431-9) in response to DNA damage and reduces the ability of p53 to induce cell cycle arrest while increasing p53-dependent apoptosis. These data suggest that PRMT5 inhibition could sensitize cells to DNA damaging agents through the induction of p53-dependent apoptosis.

In addition to directly methylating p53, PRMT5 upregulates the p53 pathway through a splicing-related mechanism. PRMT5 knockout in mouse neural progenitor cells results in the alteration of cellular splicing including isoform switching of the MDM4 gene (Bezzi M, et al. Regulation of constitutive and alternative splicing by PRMT5 reveals a role for Mdm4 pre-mRNA in sensing defects in the spliceosomal machinery. Genes Dev. 2013 Sep. 1; 27(17):1903-16). Bezzi et al. discovered that PRMT5 knockout cells have decreased expression of a long MDM4 isoform (resulting in a functional p53 ubiquitin ligase) and increased expression of a short isoform of MDM4 (resulting in an inactive ligase). These changes in MDM4 splicing result in the inactivation of MDM4, increasing the stability of p53 protein, and subsequently, activation of the p53 pathway and cell death. MDM4 alternative splicing was also observed in PRMT5 knockdown cancer cell lines. These data suggest PRMT5 inhibition could activate multiple nodes of the p53 pathway.

In addition to the regulation of cancer cell growth and survival, PRMT5 is also implicated in the epithelial-mesenchymal transition (EMT). PRMT5 binds to the transcription factor SNAIL, and serves as a critical co-repressor of E-cadherin expression; knockdown of PRMT5 results in the upregulation of E-cadherin levels (Hou Z, et al. The LIM protein AJUBA recruits protein arginine methyltransferase 5 to mediate SNAIL-dependent transcriptional repression. Mol Cell Biol. 2008 May; 28(10):3198-207).

Immunotherapies are another approach to treat hyperproliferative disorders. Enhancing anti-tumor T cell function and inducing T cell proliferation is a powerful and new approach for cancer treatment. Three immuno-oncology antibodies (e.g., immuno-modulators) are presently marketed. Anti-CTLA-4 (YERVOY/ipilimumab) is thought to augment immune responses at the point of T cell priming and anti-PD-1 antibodies (OPDIVO/nivolumab and KEYTRUDA/pembrolizumab) are thought to act in the local tumor microenvironment, by relieving an inhibitory checkpoint in tumor specific T cells that have already been primed and activated.

ICOS is a co-stimulatory T cell receptor with structural and functional relation to the CD28/CTLA-4-Ig superfamily (Hutloff, et al., “ICOS is an inducible T-cell ω-stimulator structurally and functionally related to CD28”, Nature, 397: 263-266 (1999)). Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve TH1 and TH2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natal Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both TH1 and TH2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002))

A growing body of literature supports the idea that activating ICOS on CD4+ and CD8+ effector T cells has anti-tumor potential. An ICOS-L-Fc fusion protein caused tumor growth delay and complete tumor eradication in mice with SA-1 (sarcoma), Meth A (fibrosarcoma), EMT6 (breast) and P815 (mastocytoma) and EL-4 (plasmacytoma) syngeneic tumors, whereas no activity was observed in the B16-F10 (melanoma) tumor model which is known to be poorly immunogenic (Ara G et al., “Potent activity of soluble B7RP-1-Fc in therapy of murine tumors in syngeneic hosts”, Int. J Cancer, 103(4); 501-7 (2003)). The anti-tumor activity of ICOS-L-Fc was dependent upon an intact immune response, as the activity was completely lost in tumors grown in nude mice. Analysis of tumors from ICOS-L-Fc treated mice demonstrated a significant increase in CD4+ and CD8+ T cell infiltration in tumors responsive to treatment, supporting the immunostimulatory effect of ICOS-L-Fc in these models.

Another report using ICOS^{-/-} and ICOS-L^{-/-} mice demonstrated the requirement of ICOS signalling in mediating the anti-tumor activity of an anti-CTLA4 antibody in the B16/B16 melanoma syngeneic tumor model (Fu T et al., “The ICOS/ICOSL pathway is required for optimal antitumor responses mediated by anti-CTLA-4 therapy”, Cancer Res, 71(16); 5445-54 (2011)). Mice lacking ICOS or ICOS-L had significantly decreased survival rates as compared to wild-type mice after anti-CTLA4 antibody treatment. In a separate study, B16/B16 tumor cells were transduced to overexpress recombinant murine ICOS-L. These tumors were found to be significantly more sensitive to anti-CTLA4 treatment as compared to a B16/B16 tumor cells transduced with a control protein (Allison J et al., “Combination immunotherapy for the treatment of cancer”, WO2011/041613 A2 (2009)). These studies provide evidence of the anti-tumor potential of an ICOS agonist, both alone and in combination with other immunomodulatory antibodies.

Emerging data from patients treated with anti-CTLA4 antibodies also point to the positive role of ICOS+ effector T cells in mediating an anti-tumor immune response. Patients with metastatic melanoma (Giacomo A M D et al., “Long-term survival and immunological parameters in metastatic melanoma patients who respond to ipilimumab 10 mg/kg within an expanded access program”, Cancer Immunol Immunother., 62(6); 1021-8 (2013)); urothelial (Carthon B C et al., “Preoperative CTLA-4 blockade: Tolerability and immune monitoring in the setting of a presurgical clinical trial” Clin Cancer Res., 16(10); 2861-71 (2010)); breast (Vonderheide R H et al., “Tremelimumab in combination with exemestane in patients with advanced breast cancer and treatment-associated modulation of inducible costimulator expression on patient T cells”, Clin Cancer Res., 16(13); 3485-94 (2010)); and prostate cancer which have increased absolute counts of circulating and tumor infiltrating CD4⁺ICOS⁺ and CD8⁺ICOS⁺ T cells after ipilimumab treatment have significantly better treatment related outcomes than patients where little or no increases are observed. Importantly, it was shown that ipilimumab changes the ICOS⁺ T effector:T_{reg} ratio, reversing an abundance of T_{reg}s pre-treatment to a significant abundance of T effectors vs. T_{reg}s following treatment (Liakou C I et al., “CTLA-4 blockade increases IFN-gamma producing CD4+ICOShi cells to shift the ratio of effector to regulatory T cells in cancer patients”, Proc Natl Acad Sci USA. 105(39); 14987-92 (2008)) and (Vonderheide R H et al., Clin Cancer Res., 16(13); 3485-94 (2010)). Therefore, ICOS positive T effector cells are a positive predictive biomarker of ipilimumab response which points to the potential advantage of activating this population of cells with an agonist ICOS antibody.

Though there have been many recent advances in the treatment of cancer, there remains a need for more effective and/or enhanced treatment of an individual suffering the effects of cancer.

### BRIEF DESCRIPTION OF THE DRAWINGS

FIG. 1: Four types of protein arginine methylation catalyzed by PRMTs.

FIG. 2: Known PRMT5 substrates. PRMT5 symmetrically methylates arginines in multiple proteins, preferentially in regions rich in arginine and glycine residues (Karkhanis V, et al. Versatility of PRMT5-induced methylation in growth control and development. Trends Biochem Sci. 2011 December; 36(12):633-41). The vast majority of these substrates were identified through their ability to interact with PRMT5.

FIG. 3: Molecular relationship between PRMT5/MEP50 complex activity and cyclin D1 oncogene driven pathways. MEP50, a PRMT5 coregulatory factor is a cdk4 substrate, MEP50 phosphorylation increases PRMT5/MEP50 activity. Increased PRMT5 activity mediates key events associated with cyclin D1-dependent neoplastic growth, including CUL4 (Cullin 4) repression, CDT1 overexpression, and DNA re-replication (adapted from Aggarwal P, et al. Nuclear cyclin D1/CDK4 kinase regulates CUL4 expression and triggers neoplastic growth via activation of the PRMT5 methyltransferase. Cancer Cell. 2010 Oct. 19; 18(4):329-40).

FIG. 4: Compound I C₅₀ values against PRMT5/MEP50. PRMT5/MEP50 (4 nM) activity was monitored using a radioactive assay under balanced conditions (substrate concentrations at Kₘ apparent) measuring the transfer of ³H from SAM to an H4 peptide following treatment with Compound C, Compound F, Compound B, or Compound E. IC₅₀ values were determined by fitting the data to a 3-parameter dose-response equation.

FIG. 5: The crystal structure resolved at 2.8 Å for PRMT5/MEP50 in complex with Compound C and sinefungin. The inset reveals that the compound is bound in the peptide binding pocket and makes key interactions with the PRMT5 backbone.

FIG. 6: Phylogenetic tree highlighting the methyltransferases tested in the selectivity panel. Compound C showed much greater potency for PRMT5 ( [symbol] , 10⁻⁸ M) than for any other tested enzyme ( [symbol] , >10⁻⁵ M). PRMT9 is shown for relationship purposes only within the family tree and was not evaluated in the panel. Figure adapted from Richon V M. et al.

FIG. 7: Compound C gIC₅₀ values from a 6-day growth/death assay in a panel of cancer cell lines. DLBCL-diffuse large B-cell lymphoma, GBM-glioblastoma, MCL-mantle cell lymphoma, MI-multiple myeloma

FIG. 8: Compound C gIC₁₀₀ (black squares) and Yₘᵢₙ−T0 (bars) values from a 6-day growth/death assay in a panel of cancer cell lines (top concentration used in this assay was M). DLBCL-diffuse large B-cell lymphoma, GBM-glioblastoma, MCL-mantle cell lymphoma, MI-multiple myeloma

FIG. 9: Compound B gIC₅₀ values in cancer cell lines (n=240) from 10 day 2D growth assay. ALL-acute lymphoblastic leukemia, AML-acute myeloid leukemia, CML-chronic myeloid leukemia, DLBCL-diffuse large B-cell lymphoma, HL-Hodgkin lymphoma, HN-head and neck cancer, MI-multiple myeloma, NHL-non-Hodgkin lymphoma, NSCLC-non-small cell lung cancer, PEL-primary effusion lymphoma, SCLC-small cell lung cancer, TCL-T-cell lymphoma.

FIG. 10: Compound E relative IC₅₀ values from 8-13 day colony formation assay performed in patient-derived and cell line tumor models.

FIG. 11: Compound C inhibition of SDMA. (A) A representative SDMA dose-response curve (total SDMA normalized to GAPDH) on day 3 (top) and IC₅₀ values from Z138 cells on days 1 and 3 (bottom). (B) SDMA IC₅₀ values in a panel of MCL lines (day 4).

FIG. 12: Gene expression changes in lymphoma cell lines treated with a PRMT5 inhibitor. A. Quantification of differentially expressed (DE) genes in lymphoma cell lines after Compound B (0.1 and 0.5 μM) treatment (days 2 and 4). B. Overlap of DE genes across lymphoma lines.

FIG. 13: Compound C gene expression EC₅₀ values in a panel of 11 genes identified by RNA-sequencing. Representative dose-response curves for CDKN1A (days 2 and 4, left panel) and gene panel EC₅₀ summary table (right panel, day 4).

FIG. 14: Compound B attenuates the splicing of a subset of introns in lymphoma cell lines. A. Mechanisms of regulation of cellular splicing (adapted from Bezzi M. et al.). B. Analysis of intron expression in lymphoma lines treated with 0.1 or 0.5 μM Compound B.

FIG. 15: Compound B induces isoform switching for a subset of genes in lymphoma cell lines. A. Quantification of isoform switches in 4 lymphoma cell lines treated with Compound B (0.1 and 0.5 μM) for 2 and 4 days. B. Overlap of isoform switches in 4 lymphoma lines. C. List of genes that undergo alternative splicing in all 4 lymphoma lines (overlap of 4 cell lines).

FIG. 16: MDM4 alternative splicing and p53 activation in MCL lines treated with Compound C. A. MDM4 isoform expression analysis in a panel of 4 mantle cell lymphoma lines treated with 10 and 200 nM Compound C or 5 μM Nutlin-3 for 2 and 3 days (MDM4-FL-long; MDM4-S-short). B. Western analysis of p53 and p21 expression in MCL lines treated with 10 and 200 nM Compound C or 5 μM Nutlin-3 for 3 days.

FIG. 17: Compound C induces dose-dependent changes in MDM4 RNA (A) splicing and SDMA/p53/p21 levels in Z138 cells (B).

FIG. 18: Activity of PRMT5 inhibitor and ibrutinib as single agents and in combination in MCL cell lines. A. gIC₅₀ values for Compound C and ibrutinib in a 6-day growth/death CTG assay. B. Representative growth curve for the combination of Compound B and ibrutinib in RECI cells (day 6, 1:1 ratio). C. Combination indexes (CI) for Compound B:ibrutinib in a 6-day growth/death CTG assay at the indicated ratios.

FIG. 19: Compound C efficacy and PD in a Z138 xenograft model. A. Compound C 21-day efficacy study in Z138 xenograft models. B. Quantified SDMA western data from tumors harvested at the end of the efficacy study (3 hours post last dose).

FIG. 20: Compound C efficacy and PD in a Maver-1 xenograft model. A. Compound C 21-day efficacy study in Maver-1 xenograft models. B. Quantified SDMA western data from tumors harvested at the end of the efficacy study (3 hours post last dose).

FIG. 21: Compound B growth IC₅₀ values in a panel of breast cancer cell lines from a 7-day growth 2D assay (TNBC-triple negative breast cancer, HER2-Her2 positive, HR-hormone receptor positive).

FIG. 22: Ymin-TO values from 10⁻¹² day growth/death assay in breast and MCL cell lines using the PRMT5 inhibitor, Compound C, and the PRMT5 inhibitor, Compound B.

FIG. 23: Propidium iodide FACS analysis of breast cancer lines treated with 30, 200 and 1000 nM Compound C for various periods of time (day 2, 7 and 10, biological n=2, error bars represent standard deviation).

FIG. 24: Time course of SDMA inhibition following 1 μM Compound B treatment in a panel of breast cancer cell lines. Cells were treated with DMSO or 1 μM Compound B for the indicated periods of time and cellular lysates were analyzed by western blot with SDMA and actin antibodies. The last lane on each blot is ½ of DMSO control.

FIG. 25: Compound C efficacy (left) and PK/PD (right) in a MDA-MB-468 xenograft model.

FIG. 26: 14 day growth/death CTG assay in GBM cell lines using the PRMT5 inhibitor, Compound C, and a PRMT5 inhibitor tool molecule Compound B (Ymin-TO).

FIG. 27: Compound B (1 μM) decreases SDMA levels (B), induces alternative splicing of MDM4 (A), and activates p53 (B) in GBM and lymphoma cell lines.

FIG. 28: Activity of anti-mouse ICOS agonist antibody in combination with Compound C in syngeneic tumor models. Immunocompetent mice bearing subcutaneous allografts of CT26 (colon) or EMT6 (breast) were treated with 5 mg/kg anti-ICOS (Icos17G9-GSK) and 100 mg/kg Compound C alone and in combination. Survival curves for CT26 (A) and EMT6 (B): the combination of Compound C and anti-ICOS.

### SUMMARY OF THE INVENTION

In one aspect, the present invention provides a method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding portion thereof.

In one aspect, the present invention provides a Type II protein arginine methyltransferase (Type II PRMT) inhibitor and an ICOS binding protein or antigen binding fragment thereof for use in treating cancer in a human in need thereof.

In one aspect, the present invention provides use of a Type II protein arginine methyltransferase (Type II PRMT) inhibitor and ICOS binding protein or antigen binding fragment thereof for the manufacture of a medicament to treat cancer.

In one aspect, the present invention provides use of a Type II protein arginine methyltransferase (Type II PRMT) inhibitor and ICOS binding protein or antigen binding fragment thereof for the treatment of cancer.

### DETAILED DESCRIPTION OF THE INVENTION

### Definitions

As used herein “Type II protein arginine methyltransferase inhibitor” or “Type II PRMT inhibitor” means an agent that inhibits protein arginine methyltransferase 5 (PRMT5) and/or protein arginine methyltransferase 9 (PRMT9). In some embodiments, the Type II PRMT inhibitor is a small molecule compound. In some embodiments, the Type II PRMT inhibitor selectively inhibits protein arginine methyltransferase 5 (PRMT5) and/or protein arginine methyltransferase 9 (PRMT9). In some embodiments, the Type II PRMT inhibitor is an inhibitor of PRMT5. In some embodiments, the Type II PRMT inhibitor is a selective inhibitor of PRMT5.

Arginine methyltransferases are attractive targets for modulation given their role in the regulation of diverse biological processes. It has now been found that compounds described herein, and pharmaceutically acceptable salts and compositions thereof, are effective as inhibitors of arginine methyltransferases.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modem Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et ah, Enantiomers, Racemates and Resolutions (Wiley Interscience, New York, 1981); Wilen et ah, Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

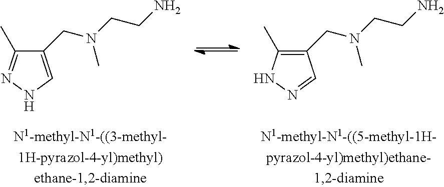

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “aliphatic,” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and subrange within the range. For example “C₁₋₆ alkyl” is intended to encompass, C₁; C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄-6, C₄-5, and C₅-6 alkyl.

“Radical” refers to a point of attachment on a particular group. Radical includes divalent radicals of a particular group.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂C₁, and the like.

“Alkenyl” refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”) In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Fused” or “ortho-fused” are used interchangeably herein, and refer to two rings that have two atoms and one bond in common, e.g.,

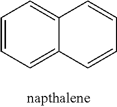

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

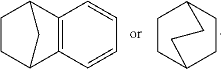

“Spiro” or “Spiro-fused” refers to a group of atoms which connect to the same atom of a carbocyclic or heterocyclic ring system (geminal attachment), thereby forming a ring, e.g.,

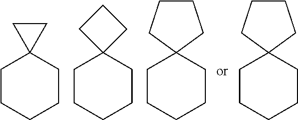

Spiro-fusion at a bridgehead atom is also contemplated.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group refers to a radical of a non-aromatic cyclic hydrocarbon group having from 3 to 10 ring carbon atoms (C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅-10 carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments,“carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅-6 cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary 5,6-bicyclic heteroaryl groups include, without limitation, any one of the following formulae:

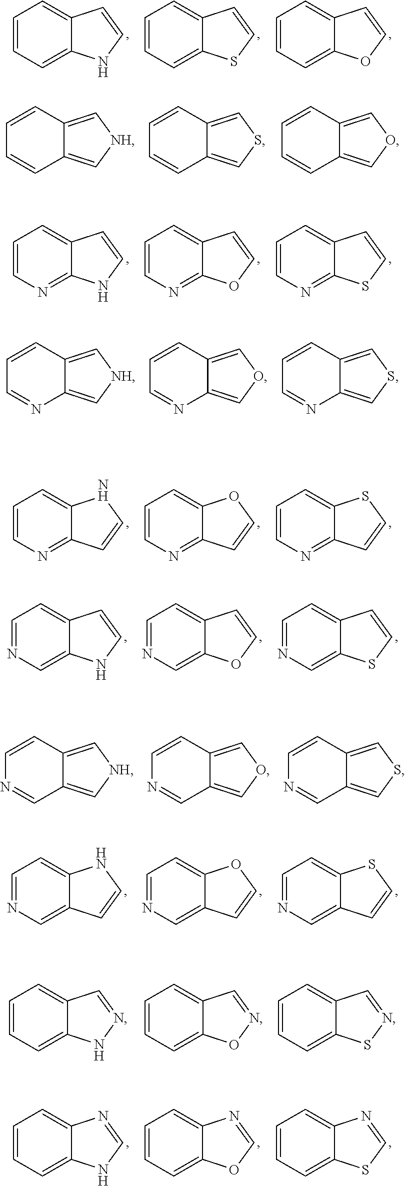

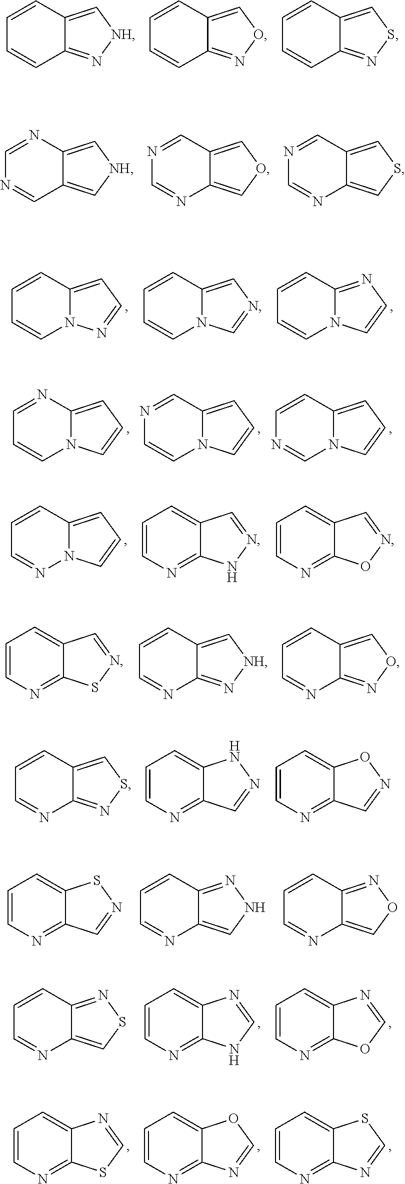

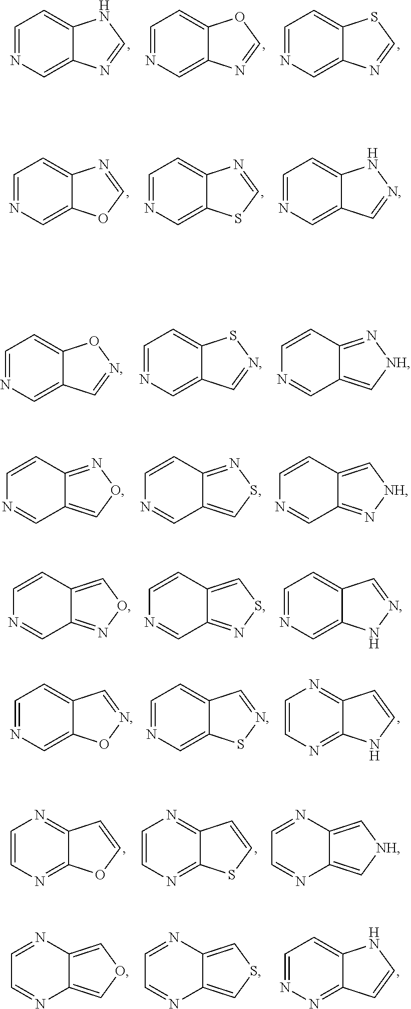

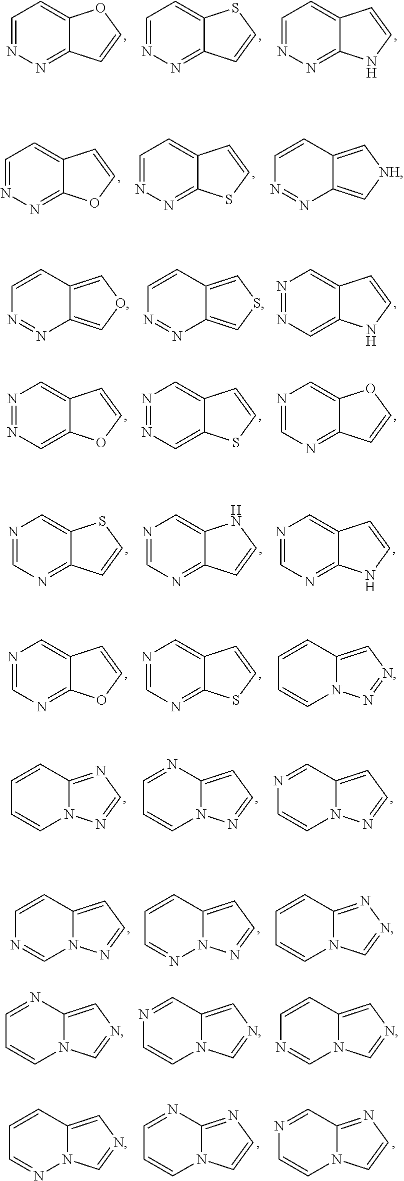

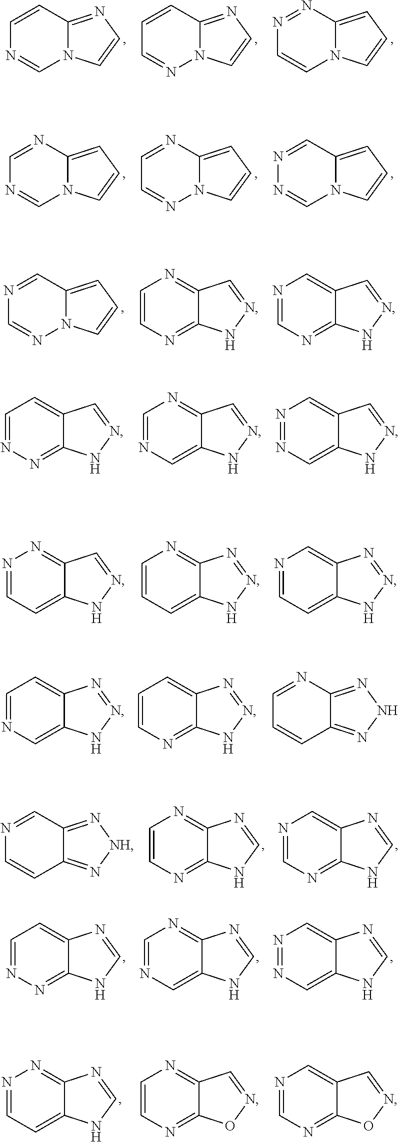

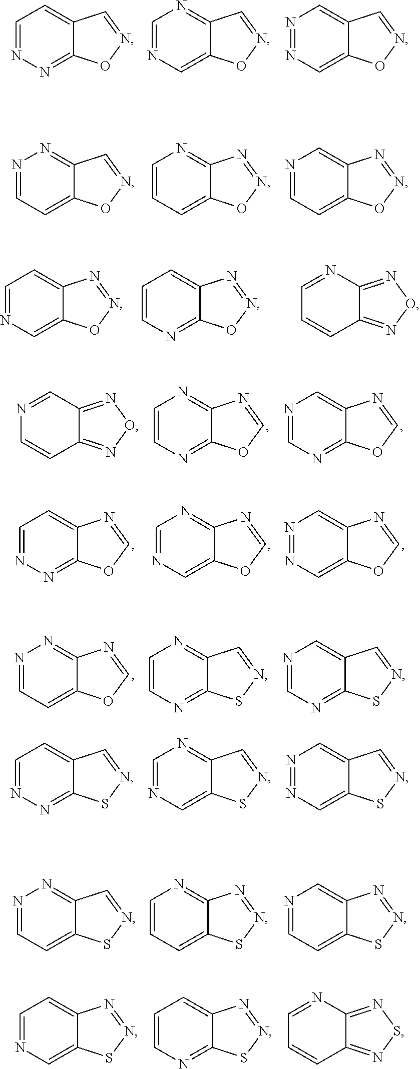

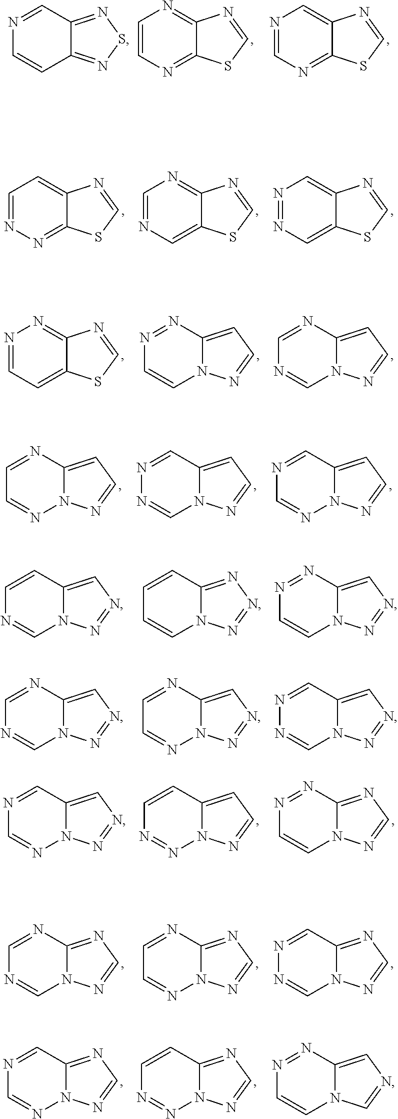

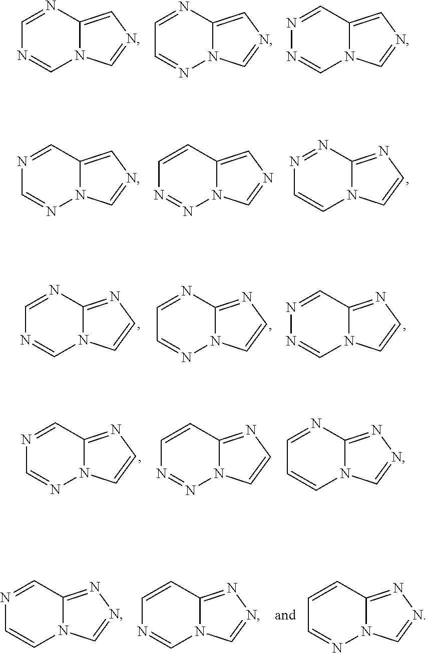

In any of the monocyclic or bicyclic heteroaryl groups, the point of attachment can be any carbon or nitrogen atom, as valency permits.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃X, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{CC})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{CC})₂, —SO₂N(R^{cc})₂, —SO₂R^{cc}, —S₂OR^{cc}, —SOR^{aa}, —C(═S)N(R^{CC})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂,—NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₁₋₆ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —C₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X, —N(OC₁₋₆ alkyl)(C₁₋₆ alkyl), —N(OH)(C₁₋₆ alkyl), —NH(OH), —SH, —S₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl),—OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂,—SO₂ C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like). “Halo” or “halogen” refers to fluorine (fluoro, —F), chlorine (chloro, —CI), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{CC})₂, —SO₂N(R^{cc})₂, —SO₂R^{cc}, —S₂OR^{cc}, —SOR^{aa}, —C(═S)N(R^{CC})₂, —C(═O)SR^{cc},—C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, RC and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(RC)₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{ff})₂, —SO₂R^{cc},—SO₂OR^{cc}, —SOR^{aa}, —C(═S)N(R^{cc})₂, —C(═O)SR^{cc}, —C(═S)SR^{CC}, C₁₋₁₀ alkyl {e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 π groups, and wherein R^{aa}, R^{bb}, RC, and R^{dd} are as defined herein. Nitrogen protecting groups are well known in the art and include those described in detail in Protecting Groups in Organic Synthesis, T. W. Greene and P. G. M. Wuts, 3 rd edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-{p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)] methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-{N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)] methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N-p-toluenesulfonylaminoacyl derivative, N-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino N-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N,N-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl] amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂ R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3 edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM),p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, l-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N,N-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂ R^{aa}, —Si(R^{aa})₃—P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group”, or “LG”, is a term understood in the art to refer to a molecular fragment that departs with a pair of electrons upon heterolytic bond cleavage, wherein the molecular fragment is an anion or neutral molecule. See, for example, Smith, March Advanced Organic Chemistry 6th ed. (501-502). Examples of suitable leaving groups include, but are not limited to, halides (such as chloride, bromide, or iodide), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, haloformates, —N02, trialkylammonium, and aryliodonium salts. In some embodiments, the leaving group is a sulfonic acid ester. In some embodiments, the sulfonic acid ester comprises the formula —OSO₂R^{LG1} wherein R^{LG1} is selected from the group consisting alkyl optionally, alkenyl optionally substituted, heteroalkyl optionally substituted, aryl optionally substituted, heteroaryl optionally substituted, arylalkyl optionally substituted, and heterarylalkyl optionally substituted. In some embodiments, R^{LG1} is substituted or unsubstituted C₁-C₆ alkyl. In some embodiments, R^{LG1} is methyl. In some embodiments, R^{LG1} is substituted or unsubstituted aryl. In some embodiments, R^{LG1} is substituted or unsubstituted phenyl. In some embodiments, R^{LG1} is:

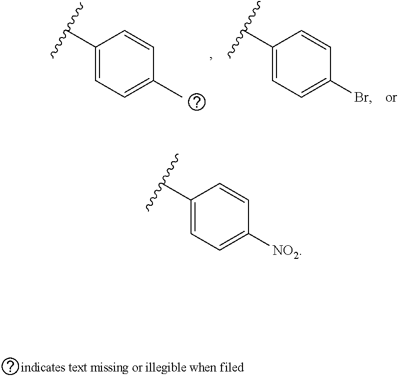

In some cases, the leaving group is toluenesulfonate (tosylate, Ts), methanesulfonate (mesylate, Ms), p-bromobenzenesulfonyl (brosylate, Bs), or trifluoromethanesulfonate (triflate, Tf). In some cases, the leaving group is a brosylate (p-bromobenzenesulfonyl). In some cases, the leaving group is a nosylate (2-nitrobenzenesulfonyl). In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66: 1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The present invention provides Type II PRMT inhibitors. In one embodiment, the Type II PRMT inhibitor is a compound of Formula (III):

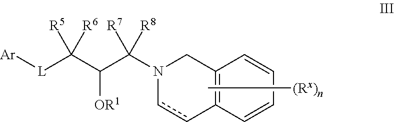

or a pharmaceutically acceptable salt thereof,

wherein

[symbol] represents a single or double bond;

R¹ is hydrogen, R^{z}, or —C(O)R^{z}, wherein R^{z} is optionally substituted C₁₋₆ alkyl;

L is —N(R)C(O)—, —C(O)N(R)—, —N(R)C(O)N(R)—,—N(R)C(O)O—, or —OC(O)N(R)—;

each R is independently hydrogen or optionally substituted C₁₋₆ aliphatic;

Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y}

groups, as valency permits;

each R^{y} is independently selected from the group consisting of halo, —CN, —NO₂, optionally substituted aliphatic, optionally substituted carbocyclyl, optionally substituted aryl,

optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;

each R^{A} is independently selected from the group consisting of hydrogen, optionally

substituted aliphatic, optionally substituted carbocyclyl, optionally substituted heterocyclyl,
optionally substituted aryl, and optionally substituted heteroaryl;

each R^{B} is independently selected from the group consisting of hydrogen, optionally

substituted aliphatic, optionally substituted carbocyclyl, optionally substituted heterocyclyl,
optionally substituted aryl, and optionally substituted heteroaryl, or two R^{B} groups are taken
together with their intervening atoms to form an optionally substituted heterocyclic ring;

R⁵, R⁶, R⁷, and R⁸ are independently hydrogen, halo, or optionally substituted aliphatic;

each R^{X} is independently selected from the group consisting of halo, —CN, optionally substituted aliphatic, —OR′, and —N(R^{ff})₂;

R′ is hydrogen or optionally substituted aliphatic;

each R″ is independently hydrogen or optionally substituted aliphatic, or two R″ are taken together with their intervening atoms to form a heterocyclic ring; and

n is 0, 1, 2, 3, 4, 5, 6, 7, 8, 9, or 10, as valency permits.

In one aspect, L is —C(O)N(R)—. In one aspect, R¹ is hydrogen. In one aspect, n is 0.

In one embodiment, the Type II PRMT inhibitor is a compound of Formula (IV):

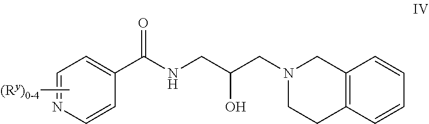

or a pharmaceutically acceptable salt thereof. In one aspect, at least one R^{y} is —NHR^{B}. In one aspect, R^{B} is optionally substituted cycloalkyl.

In one embodiment, the Type II PRMT inhibitor is a compound of Formula (VII):

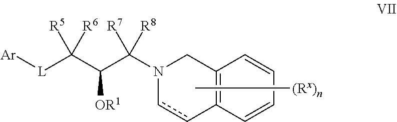

or a pharmaceutically acceptable salt thereof. In one aspect, L is —C(O)N(R)—. In one aspect, R¹ is hydrogen. In one aspect, n is 0.

In one embodiment, the Type II PRMT inhibitor is a compound of Formula (VIII):

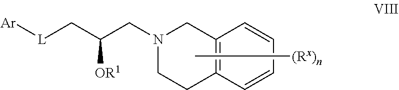

or a pharmaceutically acceptable salt thereof. In one aspect, L is —C(O)N(R)—. In one aspect, R¹ is hydrogen. In one aspect, n is 0.

In one embodiment, the Type II PRMT inhibitor is a compound of Formula (IX):

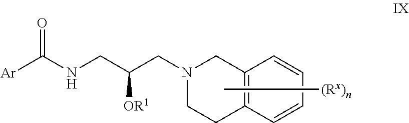

or a pharmaceutically acceptable salt thereof. In one aspect, R¹ is hydrogen. In one aspect, n is 0.

In one embodiment, the Type II PRMT inhibitor is Compound B:

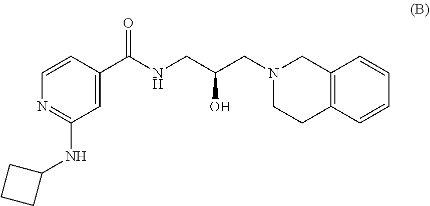 [SMILES: O=C(NC[C@H](O)CN1CCc2ccccc2C1)c1ccnc(NC2CCC2)c1]

or a pharmaceutically acceptable salt thereof.

In one embodiment, the Type II PRMT inhibitor is a compound of Formula (X):

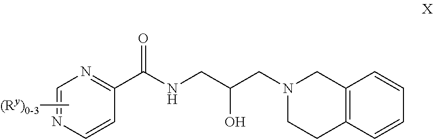

or a pharmaceutically acceptable salt thereof. In one aspect, R^{y} is —NHR^{B}. In one aspect, R^{B} is optionally substituted heterocyclyl.

In certain embodiments, the Type II PRMT inhibitor is a compound of Formula (XI):

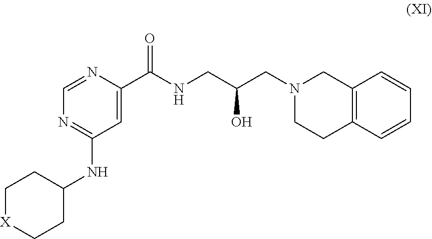

or a pharmaceutically acceptable salt thereof, wherein X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl.

In one embodiment, the Type II PRMT inhibitor is Compound C:

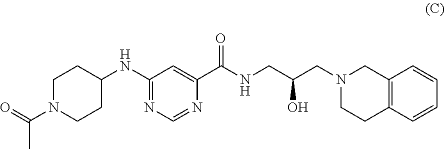 [SMILES: CC(=O)N1CCC(Nc2cc(C(=O)NC[C@H](O)CN3CCc4ccccc4C3)ncn2)CC1]

or a pharmaceutically acceptable salt thereof. Compound C and methods of making Compound C are disclosed in PCT/US2013/077235, in at least page 141 (Compound 208) and page 291, paragraph [00464] to page 294, paragraph [00469].

In another embodiment, the Type II PRMT inhibitor is Compound E:

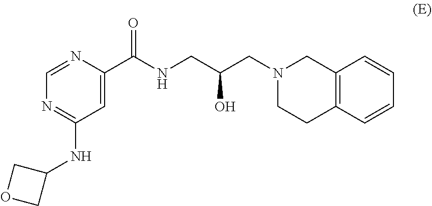 [SMILES: O=C(NC[C@H](O)CN1CCc2ccccc2C1)c1cc(NC2COC2)ncn1]

or a pharmaceutically acceptable salt thereof.

In another embodiment, the Type II PRMT inhibitor is Compound F:

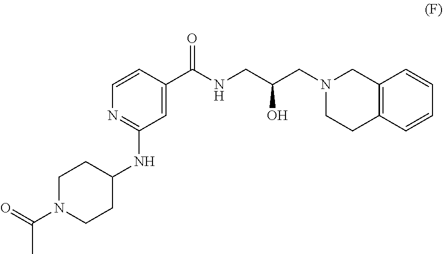 [SMILES: CC(=O)N1CCC(Nc2cc(C(=O)NC[C@H](O)CN3CCc4ccccc4C3)ccn2)CC1]

or a pharmaceutically acceptable salt thereof.

Type II PRMT inhibitors are further disclosed in PCT/US2013/077235 and PCT/US2015/043679, which are incorporated herein by reference. Exemplary Type II PRMT inhibitors are disclosed in Table 1A, Table 1B, Table 1C, Table 1D, Table 1E, Table 1F, and Table 1G of PCT/US2013/077235, and methods of making the Type II PRMT inhibitors are described in at least page 239, paragraph [00359] to page 301, paragraph [00485] of PCT/US2013/077235. Other non-limiting examples of Type II PRMT inhibitors or PRMT5 inhibitors are disclosed in the following published patent applications WO2011/079236, WO2014/100695, WO2014/100716, WO2014/100730, WO2014/100764, and WO2014/100734, and U.S. Provisional Application Nos. 62/017,097 and 62/017,055. The generic and specific compounds described in these patent applications are incorporated herein by reference and can be used to treat cancer as described herein. In some embodiments, the Type II PRMT inhibitor is a nucleic acid (e.g., a siRNA). siRNAs against PRMT5 are described for instance in Mol Cancer Res. 2009 April; 7(4): 557-69, and Biochem J. 2012 Sep. 1; 446(2):235-41.

“Antigen Binding Protein (ABP)” means a protein that binds an antigen, including antibodies or engineered molecules that function in similar ways to antibodies. Such alternative antibody formats include triabody, tetrabody, miniantibody, and a minibody. Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP may comprise the VH regions of the invention formatted into a full length antibody, a (Fab′)₂ fragment, a Fab fragment, a bi-specific or biparatopic molecule or equivalent thereof (such as scFV, bi- tri- or tetra-bodies, Tandabs, etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The terms “ABP,” “antigen binding protein,” and “binding protein” are used interchangeably herein.

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB—Q9Y6W8-2) is shown below as SEQ ID NO:9.

(SEQ ID NO: 9)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKM

The amino acid sequence of human ICOS (isoform 1) (Accession No.: UniProtKB—Q9Y6W8-1) is shown below as SEQ ID NO:10.

(SEQ ID NO: 10)
MKSGLWYFFL FCLRIKVLTG EINGSANYEM FIFHNGGVQI
LCKYPDIVQQ FKMQLLKGGQ ILCDLTKTKG SGNTVSIKSL
KFCHSQLSNN SVSFFLYNLD HSHANYYFCN LSIFDPPPFK
VTLTGGYLHI YESQLCCQLK FWLPIGCAAF VVVCILGCIL
ICWLTKKKYS SSVHDPNGEY MFMRAVNTAK KSRLTDVTL

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve TH1 and TH2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natl Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both TH1 and TH2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO 2012/131004, US20110243929, and US20160215059. US20160215059 is incorporated by reference herein. CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585. Agonist antibodies to ICOS or ICOS binding proteins are disclosed in WO2012/13004, WO2014/033327, WO2016/120789, US20160215059, and US20160304610. Exemplary antibodies in US2016/0304610 include 37A10S713. Sequences of 37A10S713 are reproduced below as SEQ ID NOS: 11-18.

37A10S713 heavy chain variable region:
(SEQ. ID NO: 11)
EVQLVESGG LVQPGGSLRL SCAASGFTFS DYWMDWVRQA
PGKGLVWVSN IDEDGSITEY SPFVKGRFTI SRDNAKNTLY
LQMNSLRAED TAVYYCTRWG RFGFDSWGQG TLVTVSS
37A10S713 light chain variable region:
(SEQ. ID NO: 12)
DIVMTQSPDS LAVSLGERAT INCKSSQSLL SGSFNYLTWY
QQKPGQPPKL LIFYASTRHT GVPDRFSGSG SGTDFTLTIS
SLQAEDVAVY YCHHHYNAPP TFGPGTKVDI K
37A10S713 V_{H} CDR1:
(SEQ. ID NO: 13)
GFTFSDYWMD
37A10S713 V_{H} CDR2:
(SEQ. ID NO: 14)
NIDEDGSITEYSPFVKG
37A10S713 V_{H} CDR3:
(SEQ. ID. NO: 15)
WGRFGFDS
37A10S713 V_{L} CDR1:
(SEQ. ID NO: 16)
KSSQSLLSGSFNYLT
37A10S713 V_{L} CDR2:
(SEQ. ID NO: 17)
YASTRHT
37A10S713 V_{L} CDR3:
(SEQ. ID NO: 18)
HHHYNAPPT

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS antigen binding protein.” Thus, as is understood in the art, anti-ICOS antibodies and/or ICOS antigen binding proteins would be considered ICOS binding proteins. As used herein, “antigen binding protein” is any protein, including but not limited to antibodies, domains and other constructs described herein, that binds to an antigen, such as ICOS. As used herein “antigen binding portion” of an ICOS binding protein would include any portion of the ICOS binding protein capable of binding to ICOS, including but not limited to, an antigen binding antibody fragment.

In one embodiment, the ICOS antibodies of the present invention comprise any one or a combination of the following CDRs:

| (SEQ ID NO: 1) | |
|---|---|
| CDRH1: | DYAMH |
| (SEQ ID NO: 2) | |
| CDRH2: | LISIYSDHTNYNQKFQG |
| (SEQ ID NO: 3) | |
| CDRH3: | NNYGNYGWYFDV |
| (SEQ ID NO: 4) | |
| CDRL1: | SASSSVSYMH |
| (SEQ ID NO: 5) | |
| CDRL2: | DTSKLAS |
| (SEQ ID NO: 6) | |
| CDRL3: | FQGSGYPYT |

In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

Humanized Heavy Chain (V_{H}) Variable Region (H2):
(SEQ ID NO: 7)
QVQLVQSGAE VKKPGSSVKV SCKASGYTFT DYAMHWVRQA
PGQGLEWMGL ISIYSDHTNY NQKFQGRVTI TADKSTSTAY
MELSSLRSED TAVYYCGRNN YGNYGWYFDV WGQGTTVTVS S

In one embodiment of the present invention the ICOS antibody comprises CDRL1 (SEQ ID NO:4), CDRL2 (SEQ ID NO:5), and CDRL3 (SEQ ID NO: 6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

Humanized Light Chain (V_{L}) Variable Region (L5)
(SEQ ID NO: 8)
EIVLTQSPAT LSLSPGERAT LSCSASSSVS YMHWYQQKPG
QAPRLLIYDT SKLASGIPAR FSGSGSGTDY TLTISSLEPE
DFAVYYCFQG SGYPYTFGQG TKLEIK

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention the ICOS antibody is an IgG4 isotype. In one embodiment, the ICOS antibody comprises an IgG4 Fc region comprising the replacement S228P and L235E may have the designation IgG4PE.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

As used herein an “immuno-modulator” or “immuno-modulatory agent” refers to any substance including monoclonal antibodies that affects the immune system. In some embodiments, the immuno-modulator or immuno-modulatory agent upregulates the immune system. Immuno-modulators can be used as anti-neoplastic agents for the treatment of cancer. For example, immuno-modulators include, but are not limited to, anti-PD-1 antibodies (Opdivo/nivolumab and Keytruda/pembrolizumab), anti-CTLA-4 antibodies such as ipilimumab (YERVOY), anti-OX40 antibodies, and anti-ICOS antibodies.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a ω-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decreases, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, V_{L}, domain antibody (dAb^{TM})), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see, e.g., Holliger and Hudson, Nature Biotechnology, 2005, Vol 23, No. 9, 1126-1136).

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

The term “domain” refers to a folded protein structure which retains its tertiary structure independent of the rest of the protein. Generally, domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb^{(TM)}” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH₁, CH₂, CH₃, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the Biacore™ methods described in Example 5 may be used to measure binding affinity.

Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein. “CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

In one aspect, a Type II protein arginine methyltransferase (Type II PRMT) inhibitor and an ICOS binding protein or antigen binding fragment thereof for use in treating cancer in a human in need thereof, is provided.

In another aspect, a method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding portion thereof, is provided.

In still another aspect, use of a Type II protein arginine methyltransferase (Type II PRMT) inhibitor and ICOS binding protein or antigen binding fragment thereof for the manufacture of a medicament to treat cancer, is provided.

In another aspect, use of a Type II protein arginine methyltransferase (Type II PRMT) inhibitor and ICOS binding protein or antigen binding fragment thereof for the treatment of cancer, is provided.

In one aspect, the present invention provides a pharmaceutical composition comprising a therapeutically effective amount of a Type II protein arginine methyltransferase (Type II PRMT) inhibitor and a second pharmaceutical composition comprising a therapeutically effective amount of an ICOS binding protein or antigen binding fragment thereof.

In another aspect, the present invention provides a pharmaceutical composition comprising a therapeutically effective amount of a Type II protein arginine methyltransferase (Type II PRMT) inhibitor and an ICOS binding protein or antigen binding fragment thereof.

In still another aspect, the present invention provides a combination of a Type II protein arginine methyltransferase (Type II PRMT) inhibitor and an ICOS binding protein or antigen binding fragment thereof.

In another aspect, a product containing a Type II PRMT inhibitor and an anti-ICOS antibody or antigen binding fragment thereof as a combined preparation for use in treating cancer in a human subject is provided.

In one embodiment, the ICOS binding protein or antigen binding fragment thereof is an anti-ICOS antibody or antigen binding fragment thereof. In another embodiment, the ICOS binding protein or antigen binding fragment thereof is an ICOS agonist. In one embodiment, the ICOS binding protein or antigen binding fragment thereof comprises one or more of: CDRH1 as set forth in SEQ ID NO: 1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In another embodiment, the ICOS binding protein or antigen binding portion thereof comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises a heavy chain variable region comprising SEQ ID NO:1; SEQ ID NO:2; and SEQ ID NO:3 and a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In another embodiment, the ICOS binding protein or antigen binding portion thereof comprises a scaffold selected from human IgG1 isotype and human IgG4 isotype. In another embodiment, the ICOS binding protein or antigen binding portion thereof comprises an hIgG4PE scaffold. In one embodiment, the ICOS binding protein is a monoclonal antibody. In another embodiment, the ICOS binding protein is a humanized monoclonal antibody. In one embodiment, the ICOS binding protein is a fully human monoclonal antibody.

In one embodiment, the Type II PRMT inhibitor is a protein arginine methyltransferase 5 (PRMT5) inhibitor or a protein arginine methyltransferase 9 (PRMT9) inhibitor. In one embodiment, the Type II PRMT inhibitor is a compound of Formula III, IV, VII, VIII, IX, X, or XI. In another embodiment, the Type II PRMT inhibitor is Compound B. In one embodiment, the Type II PRMT inhibitor is Compound C.

In one aspect, the present invention provides a Type II protein arginine methyltransferase (Type II PRMT) inhibitor and ICOS binding protein or antigen binding fragment thereof for use in treating cancer in a human in need thereof, wherein the Type II PRMT inhibitor is Compound C or a pharmaceutically acceptable salt thereof, and the ICOS binding fragment or antigen binding fragment thereof comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

In another aspect, the present invention provides a Type II protein arginine methyltransferase (Type II PRMT) inhibitor and ICOS binding protein or antigen binding fragment thereof for use in treating cancer in a human in need thereof, wherein the Type II PRMT inhibitor is Compound C or a pharmaceutically acceptable salt thereof, and the ICOS binding protein or antigen binding portion thereof comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS.

In one aspect, a method of treating cancer in a human in need thereof is provided, the method comprising administering to the human a therapeutically effective amount of a s Type II protein arginine methyltransferase (Type II PRMT) inhibitor and administering to the human a therapeutically effective amount of an ICOS binding protein or antigen binding fragment thereof, wherein the Type II PRMT inhibitor is Compound C or a pharmaceutically acceptable salt thereof, and the ICOS binding protein or antigen binding fragment thereof comprises one or more of CDRH1 as set forth in SEQ ID NO: 1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

In another aspect, a method of treating cancer in a human in need thereof is provided, the method comprising administering to the human a therapeutically effective amount of Type II protein arginine methyltransferase (Type II PRMT) inhibitor and administering to the human a therapeutically effective amount of an ICOS binding protein or antigen binding fragment thereof, wherein the Type II PRMT inhibitor is Compound C or a pharmaceutically acceptable salt thereof, and the ICOS binding protein or antigen binding portion thereof comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS.

In another aspect, a method of treating cancer in a human in need thereof is provided, the method comprising administering to the human a therapeutically effective amount of Type II protein arginine methyltransferase (Type II PRMT) inhibitor and administering a therapeutically effective amount of ibrutinib to the human. In one embodiment, the Type II PRMT inhibitor is a PRMT5 inhibitor. In one embodiment, the type II PRMT inhibitor is Compound C.

In one embodiment, the cancer is a solid tumor or a haematological cancer. In one embodiment, is melanoma, breast cancer, lymphoma, or bladder cancer.

In one embodiment the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In one aspect, the methods of the present invention further comprise administering at least one neo-plastic agent to said human.

In one embodiment the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, one or more components of a combination of the invention are administered intravenously. In one embodiment, one or more components of a combination of the invention are administered orally. In another embodiment, one or more components of a combination of the invention are administered intratumorally. In another embodiment, one or more components of a combination of the invention are administered systemically, e.g., intravenously, and one or more other components of a combination of the invention are administered intratumorally. In any of the embodiments, e.g., in this paragraph, the components of the invention are administered as one or more pharmaceutical compositions.

In one embodiment, the Type II PRMT inhibitor or the ICOS binding protein or antigen binding fragment thereof is administered to the patient in a route selected from: simultaneously, sequentially, in any order, systemically, orally, intravenously, and intratumorally. In one embodiment, the Type II PRMT inhibitor is administered orally. In another embodiment, the ICOS binding protein or antigen binding fragment thereof is administered intravenously.

In one embodiment, the methods of the present invention further comprise administering at least one neo-plastic agent to said human. The methods of the present invention may also be employed with other therapeutic methods of cancer treatment.

Typically, any anti-neoplastic agent that has activity versus a susceptible tumor being treated may be ω-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents such as diterpenoids and vinca alkaloids; platinum coordination complexes; alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents such as genetically modified T cells.

Examples of a further active ingredient or ingredients for use in combination or ω-administered with the present methods or combinations are anti-neoplastic agents. Examples of anti-neoplastic agents include, but are not limited to, chemotherapeutic agents; immuno-modulatory agents; immuno-modulators; and immunostimulatory adjuvants.

### EXAMPLES

The following examples illustrate various non-limiting aspects of this invention.

### Example 1

### Background

### PRMT5 is a Symmetric Protein Arginine Methyltransferase

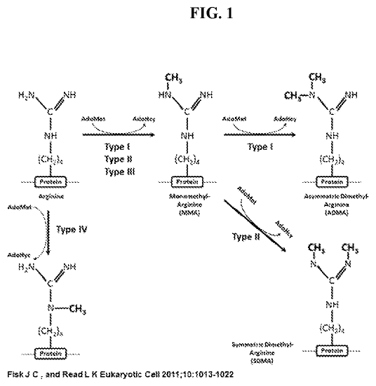

Protein arginine methyltransferases (PRMTs) are a subset of enzymes that methylate arginines in proteins that contain regions rich in glycine and arginine residues (GAR motifs). The PRMTs are categorized into four sub-types (Type I-IV) based on the product of the enzymatic reaction (FIG. 1, Fisk J C, et al. A type III protein arginine methyltransferase from the protozoan parasite Trypanosoma brucei. J Biol Chem. 2009 Apr. 24; 284(17):11590-600). Type I-III enzymes generate ω-N-monomethyl-arginine (MMA). The largest subtype, Type I (PRMT1, 3, 4, 6 and 8), progresses MMA to asymmetric dimethyl arginine (ADMA), while Type II generates symmetric dimethyl arginine (SDMA). While PRMT9/FBXO11 can also generate SDMA, PRMT5 is the primary enzyme responsible for symmetric dimethylation. PRMT5 functions in several types of complexes in the cytoplasm and the nucleus and binding partners of PRMT5 are required for substrate recognition and selectivity. Methylosome protein 50 (MEP50) is a known cofactor of PRMT5 that is required for PRMT5 binding and activity towards histones and other substrates (Ho M C, et al. Structure of the arginine methyltransferase PRMT5-MEP50 reveals a mechanism for substrate specificity. PLoS One. 2013; 8(2)).

### PRMT5 Substrates

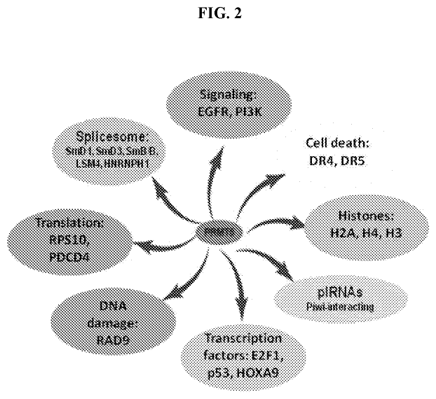

PRMT5 methylates arginines in various cellular proteins including splicing factors, histones, transcription factors, kinases and others (FIG. 2) (Karkhanis V, et al. Trends Biochem Sci. 2011 December; 36(12):633-41). Methylation of multiple components of the spliceosome is a key event in spliceosome assembly and the attenuation of PRMT5 activity through knockdown or gene knockout leads to disruption of cellular splicing (Bezzi M, et al. Regulation of constitutive and alternative splicing by PRMT5 reveals a role for Mdm4 pre-mRNA in sensing defects in the spliceosomal machinery. Genes Dev. 2013 Sep. 1; 27(17):1903-16). PRMT5 also methylates histone arginine residues (H3R8, H2AR3 and H4R3) and these histone marks are associated with transcriptional silencing of tumor suppressor genes, such as RB and ST7 (Wang L, et al. Protein arginine methyltransferase 5 suppresses the transcription of the RB family of tumor suppressors in leukemia and lymphoma cells. Mol Cell Biol. 2008 October; 28(20):6262-77; Pal S, et al. Low levels of miR-92b/96 induce PRMT5 translation and H3R8/H4R3 methylation in mantle cell lymphoma. EMBO J. 2007 Aug. 8; 26(15):3558-69). Additionally, symmetric dimethylation of H2AR3 has been implicated in the silencing of differentiation genes in embryonic stem cells (Tee W W, et al. Prmt5 is essential for early mouse development and acts in the cytoplasm to maintain ES cell pluripotency. Genes Dev. 2010 Dec. 15; 24(24):2772-7). PRMT5 also plays a role in cellular signaling, through the methylation of EGFR and PI3K (Hsu J M, et al. Crosstalk between Arg 1175 methylation and Tyr 1173 phosphorylation negatively modulates EGFR-mediated ERK activation. Nat Cell Biol. 2011 February; 13(2):174-81; Wei T Y, Juan C C, Hisa J Y, Su L J, Lee Y C, Chou H Y, Chen J M, Wu Y C, Chiu S C, Hsu C P, Liu K L, Yu C T. Protein arginine methyltransferase 5 is a potential oncoprotein that upregulates G1 cyclins/cyclin-dependent kinases and the phosphoinositide 3-kinase/AKT signaling cascade. Cancer Sci. 2012 September; 103(9):1640-50.). The role of PRMT5 in the methylation of proteins involved in cancer-relevant pathways is described below.

### PRMT5 Knockout Models

Complete loss of PRMT5 is embryonic lethal. PRMT5 plays a critical role in embryonic development which is demonstrated by the fact that PRMT5-null mice die between embryonic days 3.5 and 6.5 (Tee W W, et al. Prmt5 is essential for early mouse development and acts in the cytoplasm to maintain ES cell pluripotency. Genes Dev. 2010 Dec. 15; 24(24):2772-7). Early studies suggest that PRMT5 plays an important role in HSC (hematopoietic stem cells) and NPC (neural progenitor cells) development. Knockdown of PRMT5 in human cord blood CD34+ cells leads to increased erythroid differentiation (Liu F, et al. JAK2V617F-mediatedphosphorylation of PRMT5 downregulates its methyltransferase activity and promotes myeloproliferation. Cancer Cell. 2011 Feb. 15; 19(2):283-94). In NPCs, PRMT5 regulates neural differentiation, cell growth and survival (Bezzi M, et al. Regulation of constitutive and alternative splicing by PRMT5 reveals a role for Mdm4 pre-mRNA in sensing defects in the spliceosomal machinery. Genes Dev. 2013 Sep. 1; 27(17):1903-16).

### PRMT5 in Cancer

Increasing evidence suggests that PRMT5 is involved in tumorigenesis. PRMT5 protein is overexpressed in a number of cancer types, including lymphoma, glioma, breast and lung cancer and PRMT5 overexpression alone is sufficient to transform normal fibroblasts (Pal S, et al. Low levels of miR-92b/96 induce PRMT5 translation and H3R8/H4R3 methylation in mantle cell lymphoma. EMBO J. 2007 Aug. 8; 26(15):3558-69.; Ibrahim R, et al. Expression of PRMT5 in lung adenocarcinoma and its significance in epithelial-mesenchymal transition. Hum Pathol. 2014 July; 45(7):1397-405; Powers M A, et al. Protein arginine methyltransferase 5 accelerates tumor growth by arginine methylation of the tumor suppressor programmed cell death 4. Cancer Res. 2011 Aug. 15; 71(16):5579-87; Yan F, et al. Genetic validation of the protein arginine methyltransferase PRMT5 as a candidate therapeutic target in glioblastoma. Cancer Res. 2014 Mar. 15; 74(6):1752-65). Knockdown of PRMT5 often leads to a decrease in cell growth and survival in cancer cell lines. In breast cancer, high PRMT5 expression, together with high PDCD4 (programmed cell death 4) levels predict overall poor survival (Powers M A, et al. Protein arginine methyltransferase 5 accelerates tumor growth by arginine methylation of the tumor suppressor programmed cell death 4. Cancer Res. 2011 Aug. 15; 71(16):5579-87). PRMT5 methylates PDCD4 altering tumor-related functions. Co-expression of PRMT5 and PDCD4 in an orthotopic model of breast cancer promotes tumor growth. High expression of PRMT5 in glioma is associated with high tumor grade and overall poor survival and PRMT5 knockdown provides a survival benefit in an orthotopic glioblastoma model (Yan F, et al. Genetic validation of the protein arginine methyltransferase PRMT5 as a candidate therapeutic target in glioblastoma. Cancer Res. 2014 Mar. 15; 74(6):1752-65). Increased PRMT5 expression and activity contribute to silencing of several tumor suppressor genes in glioma cell lines.

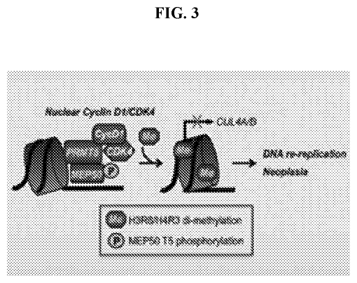

The strongest mechanistic link currently described between PRMT5 and cancer is in mantle cell lymphoma (MCL). PRMT5 is frequently overexpressed in MCL and is highly expressed in the nuclear compartment where it increases the levels of histone methylation and silences a subset of tumor suppressor genes. Recent studies uncovered the role of miRNAs in the upregulation of PRMT5 expression in MCL. More than 50 miRNAs are predicted to anneal to the 3′ untranslated region of PRMT5 mRNA. It was reported that miR-92b and miR-96 levels inversely correlate with PRMT5 levels in MCL and that the downregulation of these miRNAs in MCL cells results in the upregulation PRMT5 protein levels. Cyclin D1, the oncogene that is translocated in the vast majority of MCL patients, associates with PRMT5 and through a cdk4-dependent mechanism increases PRMT5 activity (FIG. 3, Aggarwal P, et al. Cancer Cell. 2010 Oct. 19; 18(4):329-40). PRMT5 mediates the suppression of key genes that negatively regulate DNA replication allowing for cyclin D1-dependent neoplastic growth. PRMT5 knockdown inhibits cyclin D1-dependent cell transformation causing death of tumor cells. These data highlight the important role of PRMT5 in MCL and suggest that PRMT5 inhibition could be used as a therapeutic strategy in MCL.

In other tumor types, PRMT5 has been postulated to play a role in differentiation, cell death, cell cycle progression, cell growth and proliferation. While the primary mechanism linking PRMT5 to tumorigenesis is unknown, emerging data suggest that PRMT5 contributes to regulation of gene expression (histone methylation, transcription factor binding, or promoter binding), alteration of splicing, and signal transduction. PRMT5 methylation of the transcription factor E2F1 decreases its ability to suppress cell growth and promote apoptosis (Zheng S, et al. Arginine methylation-dependent reader-writer interplay governs growth control by E2F-1. Mol Cell. 2013 Oct. 10; 52(1):37-51). PRMT5 also methylates p53 (Jansson M, et al. Arginine methylation regulates the p53 response. Nat Cell Biol. 2008 December; 10(12):1431-9) in response to DNA damage and reduces the ability of p53 to induce cell cycle arrest while increasing p53-dependent apoptosis. These data suggest that PRMT5 inhibition could sensitize cells to DNA damaging agents through the induction of p53-dependent apoptosis.

In addition to directly methylating p53, PRMT5 upregulates the p53 pathway through a splicing-related mechanism. PRMT5 knockout in mouse neural progenitor cells results in the alteration of cellular splicing including isoform switching of the MDM4 gene (Bezzi M, et al. Regulation of constitutive and alternative splicing by PRMT5 reveals a role for Mdm4 pre-mRNA in sensing defects in the spliceosomal machinery. Genes Dev. 2013 Sep. 1; 27(17):1903-16). Bezzi et al. discovered that PRMT5 knockout cells have decreased expression of a long MDM4 isoform (resulting in a functional p53 ubiquitin ligase) and increased expression of a short isoform of MDM4 (resulting in an inactive ligase). These changes in MDM4 splicing result in the inactivation of MDM4, increasing the stability of p53 protein, and subsequently, activation of the p53 pathway and cell death. MDM4 alternative splicing was also observed in PRMT5 knockdown cancer cell lines. These data suggest PRMT5 inhibition could activate multiple nodes of the p53 pathway.

In addition to the regulation of cancer cell growth and survival, PRMT5 is also implicated in the epithelial-mesenchymal transition (EMT). PRMT5 binds to the transcription factor SNAIL, and serves as a critical ω-repressor of E-cadherin expression; knockdown of PRMT5 results in the upregulation of E-cadherin levels (Hou Z, et al. The LIM protein AJUBA recruits protein arginine methyltransferase 5 to mediate SNAIL-dependent transcriptional repression. Mol Cell Biol. 2008 May; 28(10):3198-207).

These data highlight the role of PRMT5 as a critical regulator of multiple cancer-related pathways and suggest that PRMT5 inhibitors could have broad activity in heme and solid cancers. There is a strong rationale for PRMT5 inhibitors as a therapeutic strategy in MCL, as well as breast and brain cancers. These data also underline the mechanistic rationale for the use of PRMT5 inhibitors in an appropriate cellular context to:
inhibit cyclin D1-dependent functions in MCL;
activate and modulate p53 pathway activity;
modulate E2F1-dependent cell growth and apoptotic functions;
de-repress E-cadherin expression;

Compound C is a medium molecular weight (MW=452.55) potent, selective, peptide competitive, reversible inhibitor of the PRMT5/MEP50 complex with good overall physical properties and oral bioavailability. Compound C impacts several cancer related pathways ultimately leading to potent anti-cancer activity in both in vitro and in vivo models, providing a novel therapeutic mechanism for the treatment of MCL, breast and brain cancers.

### Biochemistry

Compound C was profiled in a number of in vitro biochemical assays to characterize the potency, reversibility, selectivity, and mechanism of inhibition of PRMT5.

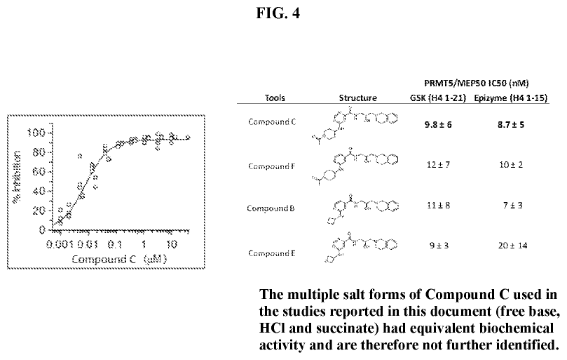

The inhibitory potency of Compound C was assessed using a radioactive assay measuring ³H transfer from SAM to a peptide derived from histone H4 identified from a histone peptide library screen. A long reaction time, 120 minutes, was used to capture any time-dependent increase in potency. Compound C was found to be a potent inhibitor of PRMT5/MEP50 with an IC₅₀ of 8.7±5 nM (n=3). This potency approaches the tight-binding limit of the assay (2 nM) and therefore represents an upper limit to the true potency of the molecule (FIG. 4). The inhibitory potency was similar for close analogs of Compound C including Compound F, Compound B and Compound E (key differences on the left hand side of the molecule) which were used as tool compounds in some biology studies.

To assess the ability of Compound C to inhibit the PRMT5 dependent methylation of cellular substrates other than histone H4, a panel of PRMT5 substrates was assembled for evaluation including SmD3, Lsm4, hnRNPH1 and FUBP1 (the majority of these substrates involved in splicing and transcriptional silencing were discovered through a cellular Methylscan™ study described below in the Biology section). Compound C effectively inhibited PRMT5/MEP50 catalyzed methylation of all of these substrates although the extremely low K_{m apparent} precluded an accurate determination of potency.

To enable interpretation of safety studies, the potency of Compound C was also evaluated against the rat and dog orthologs of the PRMT5/MEP50 complex under similar conditions as the human PRMT5 assay. Compound C potency varied <3-fold against all species (Table 2).

TABLE 2
PRMT5/MEP50 activity was monitored using a radioactive assay under
balanced conditions (substrate concentrations at K_{m apparent}) measuring
the transfer of ³H from SAM to protein substrate following treatment
with Compound C. IC₅₀ values were determined by fitting the data
to a 3-parameter dose-response equation.

| Species of | Compound C IC₅₀ |
|---|---|
| PRMT5/MEP50 | (nM) |
| Human | 9.8 ± 6 |
| Rat | 16.2 ± 5 |
| Dog | 21.2 ± 5 |

To determine the mechanism of inhibition and inhibitor binding mode, Compound C was ω-crystalized with the PRMT5/MEP50 complex and sinefungin, a natural product SAM analugue (2.8 Å resolution) (FIG. 5). The inhibitor binds in the cleft normally occupied by the substrate peptide and in close proximity to sinefungin which occupies the SAM pocket. The aryl ring of the tetrahydroisoquinoline appears to make a 7r-aryl stacking interaction with the amino group of sinefungin. A hydrogen bond is formed between the hydroxyl group of Compound C and the Leu437 backbone and Glu244. A hydrogen bond interaction is also formed between the amide of the pyrimidine ring and the backbone NH group of Phe580. The terminal piperidine acetamide lies on the solvent exposed surface with no obvious critical contacts. Overall, the structure supports an inhibitory mechanism that is uncompetitive with SAM and competitive with substrate.

To determine whether Compound C is a reversible inhibitor of PRMT5/MEP50 and to further explore the inhibitory mechanism, affinity selection mass spectrometry (ASMS) was used to measure the binding of Compound C to various PRMT5/MEP50 complexes. Positive binding could be detected in the binary complexes containing PRMT5/MEP50 with SAM, sinefungin or SAH and to the dead-end tertiary complexes of PRMT5/MEP50:H4 peptide: SAH or sinefungin. As ASMS would be unable to detect irreversibly bound Compound C, these results are consistent with a reversible binding mechanism. Upon competition with 10-fold excess H4 peptide the binding of Compound C was reduced within the PRMT5/MEP50:H4 peptide: sinefungin complex. No binding of Compound C was detected with the PRMT5/MEP50: H4 peptide complex or with PRMT5/MEP50 alone suggesting the SAM binding pocket needs to be occupied for Compound C binding. These results best fit an inhibitory mechanism that is uncompetitive with SAM and competitive with H4 peptide.

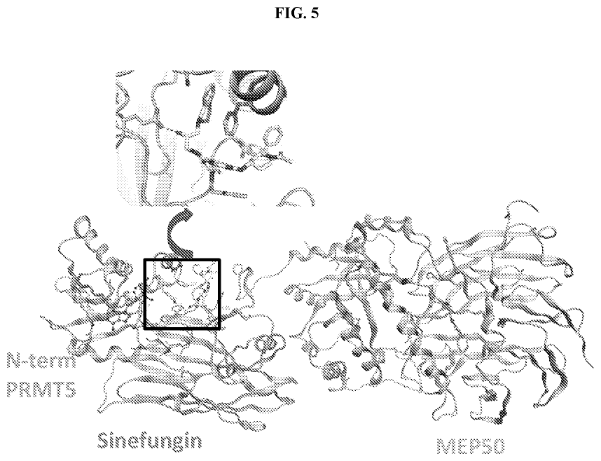

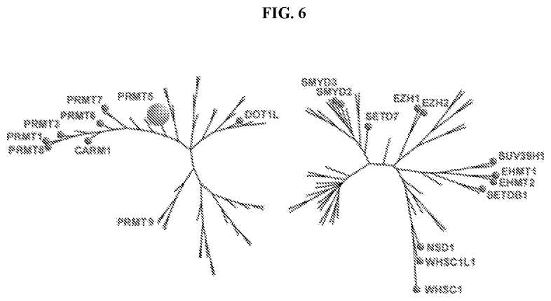

The selectivity of Compound C was assessed in a panel of enzymes that included Type I and Type II PRMTs and lysine methyltransferases (KMTs). PRMT9/FBXO11, which is the other Type II PRMT and the only PRMT to lack the THW loop, was not included due to the lack of a functional enzyme assay. Compound C did not inhibit any of the 19 enzymes on the methyltransferase selectivity panel with IC₅₀ values >40 μM resulting in >4000-fold selectivity for PRMT5/MEP50 (FIG. 6). Selectivity for PRMT5/MEP50 over the other methyltransferases was also observed for PRMT5 tool compounds that were used in the Biology section of this document (Compound B, Compound F and Compound E).

In summary, Compound C is a potent, selective, reversible inhibitor of the PRMT5/MEP50 complex with an IC₅₀ of 8.7±5 nM. The crystal structure of PRMT5/MEP50 in complex with Compound C and the ASMS binding data are consistent with a SAM uncompetitive, protein substrate competitive mechanism.

### Biology

### Summary

PRMT5 is overexpressed in a number of human cancers and is implicated in multiple cancer-related pathways. There is a strong rationale for use of PRMT5 inhibitors as a therapeutic strategy in MCL, as well as breast and brain cancers. To understand the scope of PRMT5 inhibitor anti-proliferative activity, Compound C was profiled in various in vitro and in vivo tumor models using 2D and 3D growth assays.

The identity of the genes and pathways impacted by PRMT5 inhibition are critical to understanding the mechanism of PRMT5 inhibitors required for indication prioritization, discovery of predictive biomarkers and the design of rational combination studies. Several in vitro mechanistic studies were performed to assess the biology of the response to PRMT5 inhibition. Arginine methylation levels of a number of PRMT5 substrates were assessed to monitor Compound C activity against PRMT5 in cells and xenograft tumors. RNA-sequencing of a number of cell lines was performed to evaluate the effects of Compound C on gene expression, splicing, and other molecular mechanisms and pathways that are regulated by PRMT5 activity. p53 pathway activity was monitored in cell lines treated with PRMT5 inhibitors.

Finally, Compound C activity was tested in several xenograft models of MCL and breast cancer to assess the efficacy of PRMT5 inhibition in pre-clinical cancer models and evaluate molecular mechanisms and potential biomarkers of response.

### Cell Line Sensitivity

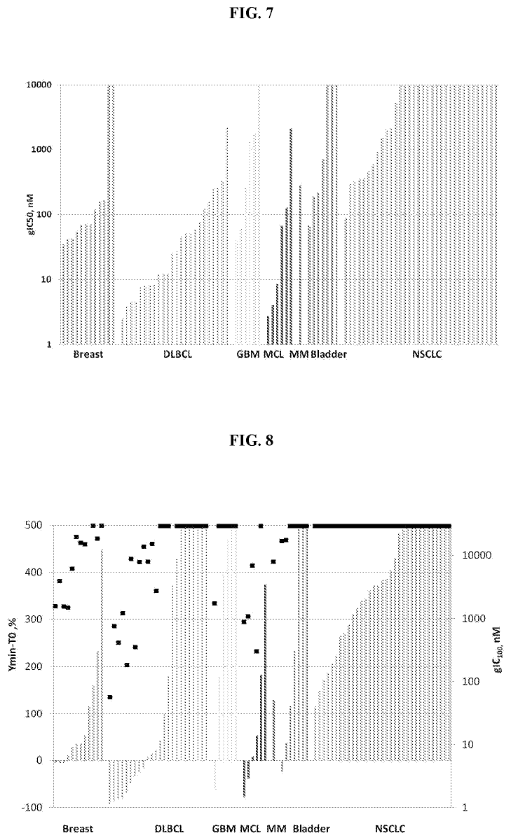

To assess the anti-proliferative activity of PRMT5 inhibition in various tumor types, Compound C was profiled in 2D and 3D in vitro assays using broad panels of cancer lines and patient-derived tumor models. First, Compound C was evaluated in a panel of cancer cell lines in a 2D 6 day growth/death assay (FIG. 7). The cell lines were selected to represent tumor types where PRMT5 activity has been reported to regulate key pathways and/or cell growth and survival (such as lymphoma and MCL, glioma, breast and lung cancer lines). Overall, the majority of cell lines tested exhibited gIC₅₀ values below 1 μM, while the most sensitive lymphoma lines (mantle cell lymphoma and diffuse large B-cell lymphoma cell lines) had gIC₅₀ values in the single digit nM range.

Compound C induced a cytotoxic response in a subset of diffuse large B-cell lymphoma (DLBCL), mantle cell lymphoma (MCL), glioblastoma, breast and bladder cancer cell lines at concentrations above 100 nM in a 6-day growth/death assay (FIG. 8, negative Ymin-T0 values). Overall, MCL and DLBLC lines exhibited the strongest cytotoxic response. The majority of breast cancer lines had low Ymin-TO values, suggesting that PRMT5 inhibition results in a complete growth inhibition in breast cancer models, while the rest of the cell lines exhibited a partial cytostatic response (positive Ymin-TO values).

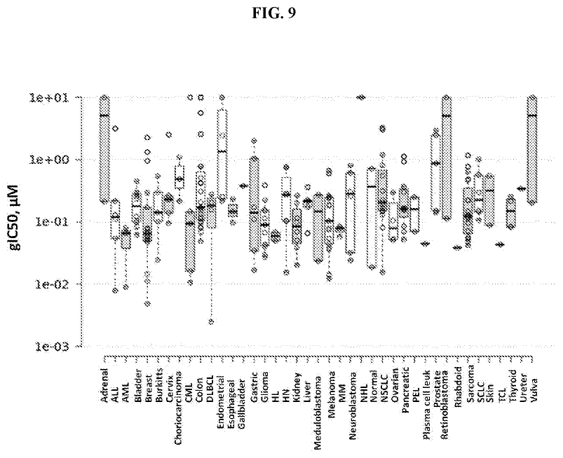

The anti-proliferative activity of PRMT5 inhibition was further tested in a large cancer cell line screen (240 cell lines, 10-day 2D growth assay) performed with a PRMT5 tool molecule (FIG. 9, biochemical/cellular activity comparison of Compound C and Compound B in FIG. 4). Overall, the majority of cell lines exhibited gIC₅₀ values lower than 1 μM. The tumor types with median gIC₅₀<100 nM were acute myeloid leukemia (AML), chronic myelogenous leukemia (CML), Hodgkin's Lymphoma (HL), multiple myeloma (MM), breast, glioma, kidney, melanoma, and ovarian cancer. These data suggest that PRMT5 inhibitors exhibit a broad range of anti-proliferative activity against various heme and solid tumor types.

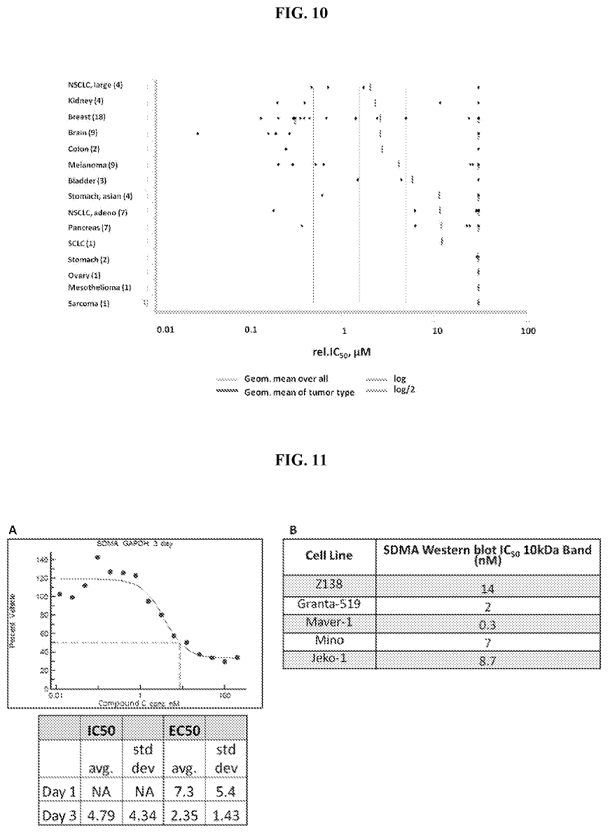

A similar broad range of anti-growth effects was observed with a PRMT5 tool compound in a panel of patient-derived tumor models and cell lines (n=73) in a soft agar 3D colony formation assay (FIG. 10). Relative growth IC₅₀ values below 1 μM were observed in 37% of the models, including tumors of non-small cell lung cancer (NSCLC), breast, melanoma, colon and glioma. Tumor types with the lowest median IC₅₀ values were large cell lung cancer, breast, kidney and glioma.

Overall, these data demonstrate that PRMT5 inhibitors have potent anti-proliferative activity in a variety of solid and hematological cancer models. The following indications were selected for additional investigation based on the activity observed in the above studies, literature hypotheses and potential for clinical development:
MCL and DLBCL (potent anti-proliferative and cytotoxic responses to PRMT5 inhibition)
Breast cancer (low gIC₅₀ values and complete growth inhibition in cell lines and low IC₅₀ values in colony formation assays in the panel of patient-derived models)
Glioblastoma (low IC₅₀ values in colony formation assay).

### Lymphoma Biology

As mentioned above, Compound C induced a potent cytotoxic response in a subset of mantle cell and diffuse large B-cell lymphoma cell lines (FIGS. 7-8). Since PRMT5 is frequently overexpressed in MCL and plays an important role in MCL pathways (such as cyclin D1 and p53), Compound C activity and mechanism were assessed in several cellular mechanistic studies. Compound C efficacy was evaluated in two xenograft models of mantle cell lymphoma.

### Cellular Mechanistic Data (Lymphoma)

### SDMA Inhibition

PRMT5 is responsible for the vast majority of cellular symmetric arginine dimethylation. To better understand the biological mechanisms linking PRMT5 inhibition to anti-cancer phenotypes, substrates were identified using an SDMA antibody recognizing a subset of cellular proteins that are symmetrically dimethylated at arginine residues. The identities of the proteins detected by the SDMA antibody were determined in Z138 cellular lysates (from control and PRMT5 inhibitor treated cells) by immunoprecipitating with the SDMA antibody and mass-spectrometric analysis (Methylscan™). Amongst SDMA containing proteins the vast majority were factors that are involved in cellular splicing and RNA processing (SmB, Lsm4, hnRNPH1 and others), transcription (FUBP1) and translation, highlighting the role of PRMT5 as an important regulator of cellular RNA homeostasis.

The SDMA antibody was then used in western and ELISA assays to measure Compound C dependent inhibition of methylation. First, Z138 MCL cells (Compound C gIC₅₀ 2.7 nM, gIC₉₅ 82 nM and gIC₁₀₀ 880 nM, cytotoxic response in a 6-day growth/death assay, FIGS. 7-8) were treated with increasing concentrations of Compound C to determine the cellular IC₅₀ of SDMA inhibition on days 1 and 3 post treatment (FIG. 11).

An SDMA ELISA revealed time-dependent changes in SDMA levels with IC₅₀ values of 4.79 nM on day 3 and EC₅₀ of 7.3 and 2.35 on days 1 and 3, respectively (FIG. 11, panel A,). Complete inhibition of SDMA was observed at concentrations above 19 nM (EC₉₀) on day 3. Complete growth inhibition in Z138 cells as observed between gIC₉₅ (82 nM) and gIC₁₀₀ (880 nM) (in a 6-day growth/death assay), concentrations that are above the EC₉₀ of SDMA inhibition. These data suggest that in order to trigger complete growth inhibition and cytotoxicity in Z138 cells, PRMT5 activity needs to be inhibited >90%.

In order to evaluate whether the inhibition of SDMA levels is predictive of cellular growth response to Compound C, SDMA IC₅₀ values were determined in a panel of MCL cell lines. SDMA IC₅₀ values were in a range of 0.3 to 14 nM in a panel of 5 MCL lines (FIG. 11, panel B) (sensitive Z138, Granta-519, Maver-1 and moderately resistant Mino, and Jeko-1, FIGS. 7-8) suggesting that SDMA is not a response marker, but rather a marker of PRMT5 activity that could be used to monitor PRMT5 inhibition in sensitive and resistant models.

### Gene Expression Profiling of Lymphoma Cell Lines

PRMT5 methylates histones and proteins involved in RNA processing and therefore PRMT5 inhibition is expected to have a profound effect on cellular mRNA homeostasis. To further decipher cellular mechanisms that are regulated by PRMT5 and contribute to the cellular response to PRMT5 inhibitors, global gene expression changes were evaluated in lymphoma models sensitive to PRMT5 inhibition. To elucidate gene expression changes that occur in lymphoma cell lines upon PRMT5 inhibitor treatment, 4 sensitive lymphoma lines (2 MCL lines-Z138 and Granta-519 and 2 DLBCL lines-DOHH2 and RL) were profiled by RNA-sequencing.

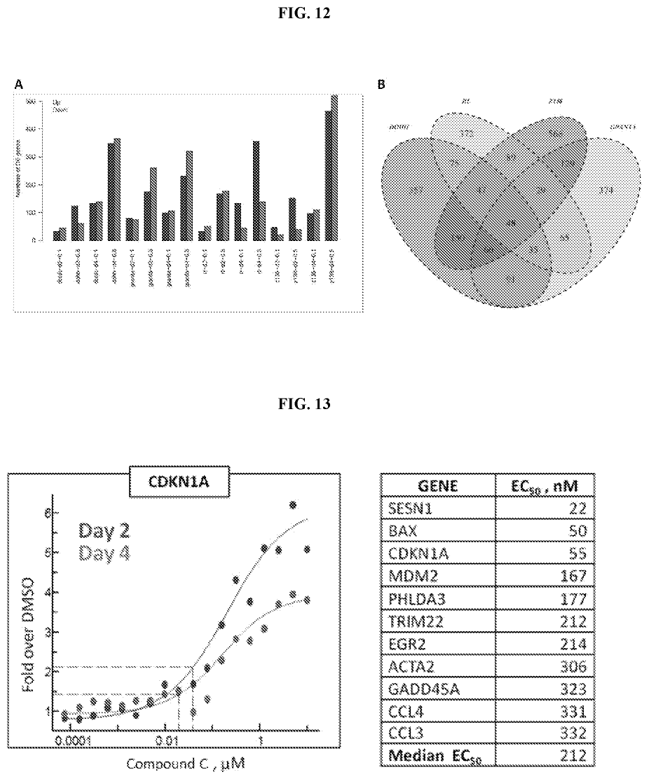

First, gene expression changes were evaluated in lymphoma lines treated with increasing concentrations of PRMT5 tool molecule for 2 and 4 days (FIG. 12). The effect on RNA expression was time- and dose-dependent and 48 genes were commonly regulated across 4 lymphoma lines. These data demonstrate that PRMT5 inhibition triggers expression changes in several hundred of genes and a subset of these changes is common for all 4 sensitive lymphoma lines tested. The relevance of these genes in the mechanism of cellular response to PRMT5 inhibition is being evaluated.

### SDMA and Gene Expression Changes

To confirm the gene expression changes discovered by the RNA-seq experiment, qPCR analysis of the expression of a subset of genes was performed (genes with robust changes and genes involved in p53 pathway). Z138 cells were treated with increasing doses of Compound C for 2 and 4 days, RNA was isolated and analyzed by qPCR. FIG. 13 shows representative dose-response curves in the left panel and gene expression EC₅₀ values (day 4) are summarized in the right panel. Overall, all 11 genes tested showed time- and dose-dependent expression changes and the EC₅₀ values were in the range of 22 to 332 nM, with a median gene expression EC₅₀ of 212 nM. Importantly, the gene expression median EC₅₀ value corresponds to the Compound C concentration that results in the maximal inhibition of cellular methylation in Z138 (as measured by SDMA antibody ELISA, FIG. 11), suggesting that near complete inhibition of PRMT5 activity is required to establish changes in the gene expression program. These data highlight the connection of the extent of PRMT5 inhibition with changes in gene expression and growth phenotypes, where both require near complete inhibition of PRMT5 activity.

### PRMT5 Inhibition and Splicing

Since PRMT5 methylates spliceosome subunits and PRMT5 inhibition attenuates arginine methylation of a number of proteins involved in splicing, the effect of PRMT5 inhibin on cellular splicing was studied. The changes in RNA splicing were assessed in the lymphoma RNA-seq dataset described above.

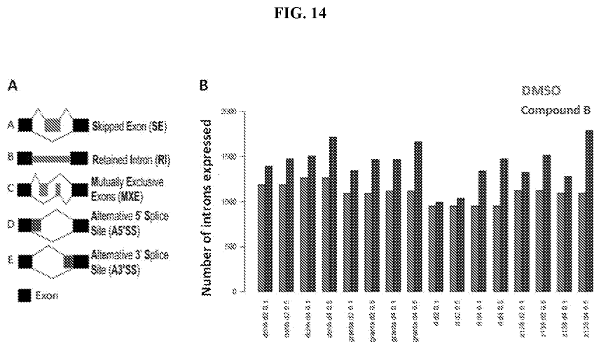

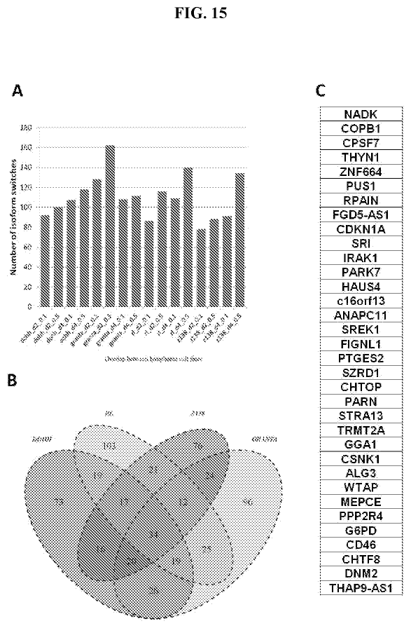

There are several molecular mechanisms by which cellular splicing might be regulated (FIG. 14, panel A), where retention of introns (B) usually results in changes of gene expression, while exon skipping or the usage of alternative splice sites lead to isoform switching (A, C-E). PRMT5 tool compound treatment resulted in a dose- and time-dependent increase of intron retention in all lymphoma lines tested (FIG. 14, panel B). Interestingly, splicing factor map analysis suggested that a subset of splicing factors binding sites were enriched at retained introns across all four cell lines, including hnRNPH1 (directly methylated by PRMT5), hnRNPF, SRSF1 and SRSF5, suggesting that PRMT5 effects on cellular splicing might be dependent on the methylation of multiple components of spliceosome machinery (Sm and hnRNP proteins). PRMT5 inhibition also induced isoform switching (alternative splicing) in lymphoma cell lines (FIG. 15, panel A) and 34 genes showed consistent alternative splicing changes across all cell lines tested (FIG. 15, panels B and C).

Overall, changes in the splicing of several hundred genes were observed, highlighting that PRMT5 effects on splicing are not global, but rather are specific to a limited number of RNAs. One likely explanation for such specificity could be that PRMT5 directly regulates RNA binding of specific splicing factors, such as hnRNPH1 and others. The role of alternative splicing changes in the mechanism of action of PRMT5 inhibitors is being explored and one particular example is discussed in the section below.

### MDM4 Splicing and Activation of the p53 Pathway

It has been reported that PRMT5 knockout or knockdown results in an MDM4 isoform switch, which leads to the inactivation of MDM4 ubiquitin ligase activity toward p53 (described in the BACKGROUND section). PRMT5 inhibition resulted in the activation of the p53 pathway in 4 lymphoma lines tested in an RNA-seq experiment (GSEA). To understand whether p53 activation is associated with MDM4 isoform switching, MDM4 alternative splicing was analyzed. The MDM4 isoform switch was observed in all 4 lymphoma lines. Next, changes in MDM4 splicing were confirmed in a panel of 4 MCL lines by RT-PCR (FIG. 16, panel A, Z138, JVM-2 and MAVER-1 MCL lines are sensitive to Compound C, while REC-1 is the most resistant MCL line). In Z138 and JVM-2 cells (both p53 wild-type) Compound C induced MDM4 isoform switching. In MAVER-1 and REC-1 cells (both p53 mutant), the basal expression of the MDM4 long form was low/undetectable and therefore, MDM4 isoform switching could not be detected. Subsequently, p53 and p21 (or CDKN1A, a p53 target gene) protein expression increased in JVM-2 and Z138 cells (FIG. 16, panel B). Importantly, in Z138 cells, 200 nM Compound C and 5 μM MDM2 inhibitor (Nutlin-3) treatment increased p53 and p21 expression to similar levels. These data suggest that PRMT5 inhibition regulates MDM4 splicing in cell lines that express high levels of the MDM4 long isoform and induces the p53 pathway activity in p53 wild-type cell lines. The role of the p53 pathway in the biology of the response of p53 wild-type MCL cells to PRMT5 inhibition is being evaluated.

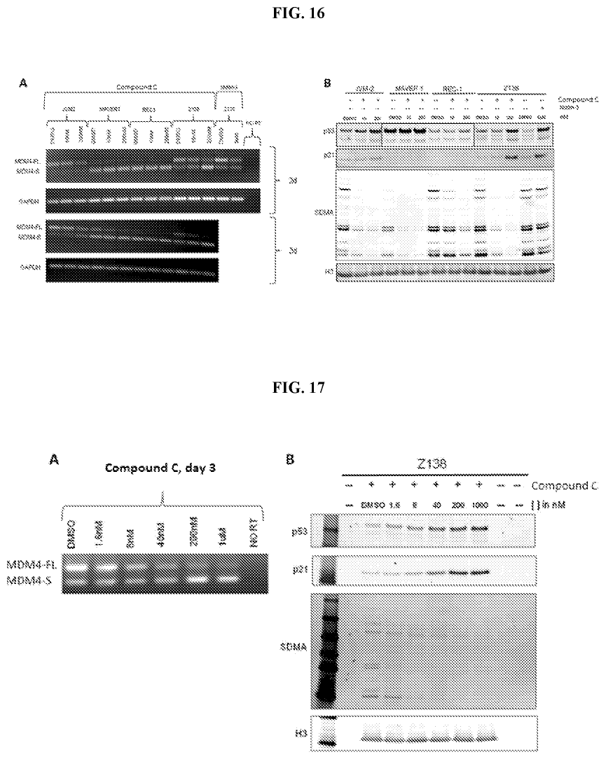

Additionally, the dose-response of changes in MDM4 splicing, SDMA inhibition and p53 expression were evaluated in Z138 cells treated with increasing concentrations of Compound C to evaluate the relationship of PRMT5 inhibition, MDM4 splicing and p53 activation (FIG. 17, panel A and B). SDMA levels were undetectable by Western blot at the concentrations of Compound C above 8 nM. At the same time, changes in MDM4 splicing and p53/p21 protein expression were apparent at concentrations of Compound C above 8 nM. These results suggest that PRMT5 activity needs to be substantially inhibited (no SDMA levels detectable by Western) before changes in gene splicing and subsequent pathway activity will occur (MDM4/p53/p21).

These data suggest that PRMT5 inhibition activates wild-type p53 through the regulation of MDM4 splicing. Such a mechanism could be useful in cancer types where p53 is not frequently mutated, such as heme and pediatric malignancies. In lymphoma models, PRMT5 inhibition leads to significant (GSEA analysis) and relatively quick activation of the p53 pathway, which likely contributes to the growth/death phenotypes observed in cell lines treated with PRMT5 inhibitor. Knockdown/rescue experiments will be used to further evaluate the role of the MDM4/p53 pathway in the PRMT5 inhibitor induced cellular responses.

MDM4 isoform expression and p53 mutation are potential predictive biomarkers of response to PRMT5 inhibition in MCL. In an MCL cell line panel, the only two wild-type p53 lines, Z138 and JVM-2, were the most sensitive lines (the lowest gIC₅₀ values and the only two MCL lines that exhibit cytotoxicity in a 6-day growth/death assay). In both cell lines, Compound C treatment led to an MDM4 isoform switch and p53 pathway activation. The limited number of MCL cell lines and extremely low success rate of the establishment of primary MCL models precludes us from further evaluation of the p53 predictive biomarker hypothesis. While the p53 pathway could be important for the biology of the response of p53 wild-type cells to PRMT5 inhibitors, our data strongly underlines the importance of other pathways that can drive anti-tumor efficacy as well, since PRMT5 inhibition results in anti-proliferative effects in the absence of functional p53 (ex. Maver-1 cell line).

### Mantle Cell Lymphoma: Comparison and Combination Activity of Compound C and Ibrutinib.

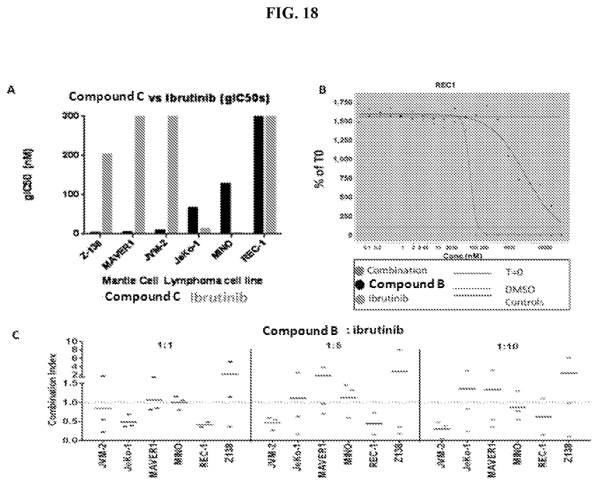

Bruton's tyrosine kinase (BTK) inhibitor ibrutinib was recently approved for use in MCL with an unprecedented overall response rate of nearly 70 percent in the relapsed/refractory setting (Wang M L, et al. N Engl J Med. 2013 Aug. 8; 369(6):507-16). The majority of patients treated with ibrutinib, however, do not achieve complete remission, and the median progression-free survival is approximately 14 months. To understand, whether Compound C could be used in ibrutinib resistant MCL, Compound C and ibrutinib sensitivity were assessed in a 6-day growth/death assay (FIG. 18, panel A). The cell lines that have low Compound C gIC₅₀ values (Z-138, Maver-1 and JVM-2) are resistant to ibrutinib, while ibrutinib sensitive lines (Mino, Jeko-1) are only moderately sensitive to Compound C (FIG. 18, panel A). This data suggests that the activity profiles of ibrutinib and Compound C do not overlap and that ibrutinib resistant MCL models are sensitive to PRMT5 inhibition. Additionally, the combination of PRMT5 inhibitor and ibrutinib demonstrated synergistic anti-proliferative activity in the majority of MCL lines tested (Combination Index (CI)<1) (FIG. 18, panels B and C), suggesting that the combination of the two compounds may provide increased therapeutic benefit. These data indicate that PRMT5 inhibitors could be used in an ibrutinib resistant MCL patient population and that the combination of PRMT5 inhibitors with ibrutinib could be explored in both ibrutinib refractory and sensitive settings.

### Efficacy in Mantle Cell Lymphoma Models

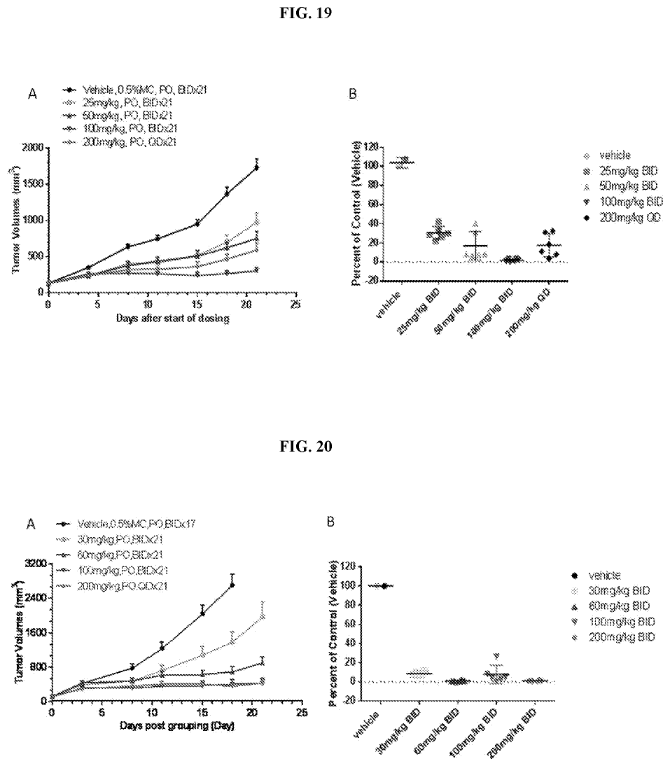

To test whether the efficacy observed in in vitro growth/death assays in lymphoma cell line models translates to an in vivo setting, Compound C efficacy studies were performed in xenograft models of mantle cell lymphoma (sensitive Z138 and Maver-1 cell lines). First, the therapeutic effects of Compound C treatment on tumor growth were tested in a 21-day efficacy study in a Z-138 MCL xenograft model. Tumors in all the Compound C dose groups showed significant differences in weight and volume compared to vehicle samples ranging from a minimum of 40% TGI at the lowest dose group (25 mg/kg BID) to as high as >90% in the top 100 mg/kg BID dose group (no body weight loss was observed in all groups in all efficacy studies presented, FIG. 19, panel A). PD analysis of tumors using the SDMA western showed that all dose groups had greater than 70% reduction of the methyl mark ranging as high as >98% in the top dose groups (FIG. 19, panel B).

Next, efficacy of Compound C was assessed in a Maver-1 MCL xenograft model (FIG. 20). Tumors in all the Compound C dose groups measured on day 18 showed significant differences in volume compared to vehicle samples ranging from a minimum of 50% TGI at the lowest dose group to as high as >90% in the top dose groups. PD analysis of tumors using SDMA showed that all dose groups had 80-95% reduction of the methyl mark.

These data demonstrate that Compound C treatment results in significant tumor growth inhibition (close to 100% TGI) in xenograft models of mantle cell lymphoma. It appears that almost complete inhibition of the SDMA signal (>90%) is required for maximal TGI (>90%), suggesting that in order to obtain significant efficacy in tumors, PRMT5 activity needs to be inhibited >90%.

### Lymphoma Biology Summary

The strongest mechanistic link currently described between PRMT5 and cancer is in MCL. PRMT5 is frequently overexpressed in MCL and is highly expressed in the nuclear compartment where it increases levels of histone methylation and silences a subset of tumor suppressor genes. Importantly, cyclin D1, the oncogene that is translocated in the vast majority of MCL patients, associates with PRMT5 and through a cdk4-dependent mechanism increases PRMT5 activity. PRMT5 mediates the suppression of key genes that negatively regulate DNA replication allowing for cyclin D1-dependent neoplastic growth. PRMT5 knockdown inhibits cyclin D1-dependent cell transformation causing death of tumor cells. These data highlight the important role of PRMT5 in MCL and suggest that PRMT5 inhibition could be used as a therapeutic strategy in MCL.
Compound C inhibits growth and induces death in MCL cell lines, which are amongst the most sensitive cell lines tested to date (in a 6-day growth/death assay). In a panel of MCL lines tested, 3 cell lines had gIC₅₀<10 nM, 2 lines exhibited gIC₅₀<100 nM and 1 cell line had gIC₅₀>1 μM. Compound C effect on the downstream targets of PRMT5 and cyclin D1 is currently being investigated to evaluate whether it contributes to the anti-growth and pro-apoptotic response.
SDMA antibody Methylscan™ was used to evaluate PRMT5 substrates in MCL lines. The vast majority of SDMA containing proteins were factors that are involved in cellular splicing and RNA processing (SmB, Lsm4, hnRNPH1 and others), transcription (FUBP1) and translation highlighting the role of PRMT5 as an important regulator of cellular RNA homeostasis. The SDMA antibody was further used to evaluate PRMT5 inhibition in a panel of MCL lines where SDMA IC₅₀ values were similar in sensitive and resistant models, suggesting that SDMA is not a marker of response but rather a marker of PRMT5 inhibition.
Compound C treatment induced splicing changes in a subset of RNAs, in particular, an MDM4 isoform switch was observed in MCL and DLBCL lines, suggesting that PRMT5 inhibition activates the p53 pathway through the regulation of MDM4 splicing. Knockdown/rescue experiments will be used to further evaluate the role of the MDM4/p53 pathway in PRMT5 inhibitor induced cellular responses.
MDM4 isoform expression and p53 mutation are potential predictive biomarkers of response to PRMT5 inhibition in MCL. In a MCL cell line panel, the two wild-type p53 lines, Z138 and JVM-2, were the most sensitive lines (the lowest gIC₅₀ values and the only two MCL lines that exhibit cytotoxicity in a 6-day growth/death assay).
In recent years, the clinical exploration of ibrutinib drastically changed the approach to MCL treatment. In vitro data indicate that PRMT5 inhibitors could be used in an ibrutinib resistant MCL patient population and that the combination of PRMT5 inhibitors with ibrutinib could be explored in both ibrutinib refractory and sensitive settings.
In vivo studies demonstrate that Compound C treatment results in significant tumor growth inhibition (close to 100% TGI) in xenograft models of mantle cell lymphoma. It appears that in order to obtain maximal efficacy in tumors (TGI >90%), almost complete inhibition of PRMT5 activity (>90%) is required.

### Breast Cancer Biology

The cell line screening data demonstrate that breast cancer cell lines are sensitive to PRMT5 inhibition and exhibit nearly complete growth inhibition in a 2D 6-day growth/death assay (low Ymin-TO, FIGS. 7-9). Additionally, the data from the colony formation assay in a panel of patient-derived (PDX) tumor models suggested that breast tumors are amongst the most sensitive tumors in the panel (based on the Compound E rel. IC₅₀ values, FIG. 10). Thus, breast cancer cell lines were assessed in several growth/death and mechanistic studies to assess the role and the therapeutic potential of PRMT5 inhibition in breast cancer.

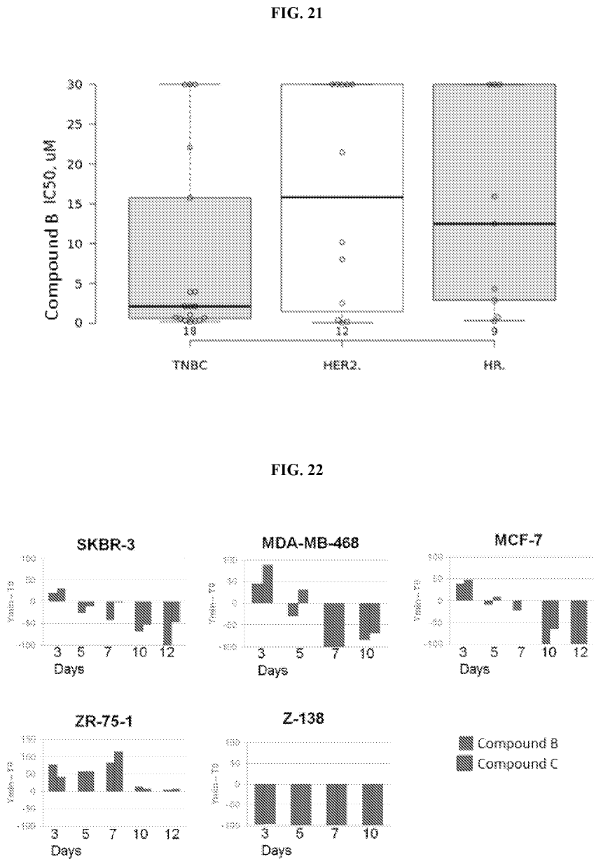

In order to understand PRMT5 inhibitor activity across different breast tumor subtypes, a panel of breast cancer cell lines was profiled in a 7-day growth assay using a PRMT5 tool compound (FIG. 21). PRMT5 inhibition attenuates cell growth with low IC₅₀ values across the various subtypes of breast cancer cell lines tested. The median IC₅₀ value was the lowest in TNBC (triple negative breast cancer) cell lines compared to the HER2 or hormone receptor (HR) positive lines.

In a 6-day growth/death assay, the majority of breast cancer cell lines exhibited cytostatic effect. To evaluate whether pro-longed exposure to Compound C will affect the cytostatic vs. cytotoxic nature of the response, PRMT5 inhibitors were evaluated in a longer-term growth/death assay (FIG. 22). In SKBR3, MDA-MB-468 and MCF-7 cells, treatment with Compound C (as well as tool molecule Compound B) led to a cytotoxic response upon prolonged exposure to compound (7-10 days). In ZR-75-1 cells, the PRMT5 inhibitors triggered a cytostatic response at all time points (days 3-12), while Z-138 (MCL, included as a control) cells exhibited profound overall net cell death at all time points (days 3-10) of the assay. These data suggest that PRMT5 inhibition leads to a net cell death (cytotoxic response) upon longer exposures (>5 days) in a subset of breast cancer cell lines.

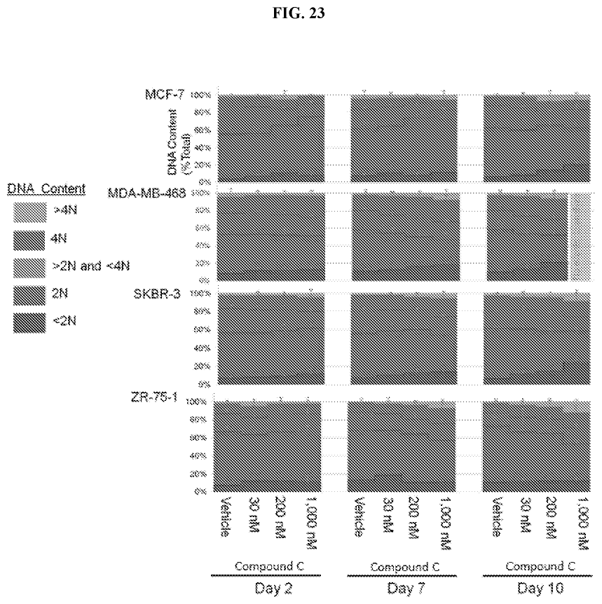

To test whether Compound C effects on cell growth were associated with changes in cell cycle distribution, the effects of Compound C on the cell cycle were evaluated using propidium iodide FACS (fluorescence activated cell sorting) analysis (FIG. 23). Overall, the FACS results are consistent with the long-term proliferation data, demonstrating that in 3 out of 4 breast cancer lines, long-term Compound C treatment resulted in the induction of cell death (increase in <2N) after 7-10 days of treatment. In MCF-7 cells (p53 wild-type), Compound C treatment led to the accumulation of cells in G1 phase (2N) and the loss of cells from S phase of the cell cycle (>2N and <4N) on day 2, with subsequent cell death as evidenced by the accumulation of cells in sub-G1 phase (<2N) on day 10. In ZR-75-1 cells (p53 wild-type), Compound C had minor effects on cell cycle distribution where there was a decrease in G1 (2N) and an increase in >4N cell fractions on days 7 and 10. MDA-MB-468 and SKBR-3 cell lines responded similarly to Compound C treatment with a decrease in G1 (2N) phase (day 7 or day 10), an increase in G2/M (4N) and >4N DNA content, which coincided with the accumulation of cells in subG1 (<2N), indicative of cell death. These data suggest that PRMT5 inhibition impacts the distribution of cells in the cell cycle and that the phenotypic outcome depends on the cellular context.

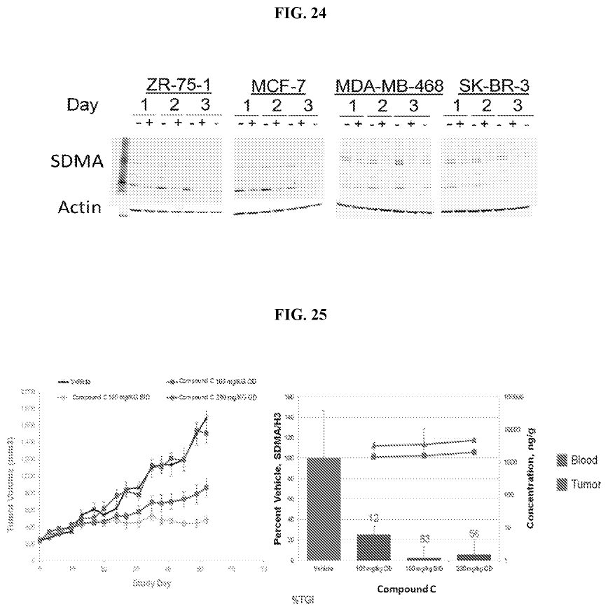

In order to evaluate whether PRMT5 activity was equally inhibited in sensitive and resistant breast cancer lines, the levels of SDMA were measured in cells following PRMT5 inhibitor treatment (FIG. 24). Overall, the timing of the SDMA decrease was similar for all cell lines tested (sensitive and resistant). The maximal inhibition of SDMA was observed on day 3. The SDMA IC₅₀ in MDA-MB-468 cells was 5.4 nM, similar to the SDMA IC₅₀ in Z138 cells. These data indicate that SDMA is a marker of PRMT5 catalytic activity and is not predictive of antiproliferative response to PRMT5 inhibition. SDMA IC₅₀ values are being further evaluated in a panel of breast cancer lines.

### Efficacy in In Vivo Breast Cancer Models

Next, the efficacy of PRMT5 inhibition was evaluated in in vivo models of breast cancer. First, MDA-MB-468, a triple negative breast cancer xenograft model, was treated with 100 mg/kg (QD and BID) and 200 mg/kg (QD) of Compound C (FIG. 25). Maximal tumor growth inhibition (TGI=83%) was observed in the 100 mg/kg BID treated group, where SDMA inhibition was greater than 90%, while in the 100 mg/kg QD treated animals, Compound C treatment was not efficacious and SDMA inhibition was less than 80%. This data suggests that the SDMA levels need to be nearly completely inhibited (>90%) in order to see significant TGI in in vivo breast cancer xenograft models.

### Breast Cancer Summary

In breast cancer, high PRMT5 expression and high PDCD4 (programmed cell death 4) levels predict overall poor survival.
Breast cancer cell lines and breast cancer patient-derived models were amongst the most sensitive models tested in a 2D growth/death and colony formation assays.
Compound C treatment resulted in complete growth inhibition in a 6-day growth/death assay and pro-longed exposure to PRMT5 inhibitor induced cell death in 3 out of 4 cell lines tested.
In a 7-day proliferation assay, TNBC cell lines were more sensitive to PRMT5 inhibition than Her2 and hormone receptor positive lines.
SDMA levels were decreased in sensitive and resistant breast cancer lines treated with PRMT5 inhibitor, suggesting that SDMA is not a marker of response but rather a marker of PRMT5 activity.
In a MDA-MB-468 xenograft model, Compound C treatment resulted in tumor growth inhibition (TGI=83%) in the 100 mg/kg BID treated group, where SDMA inhibition was greater than 90%, while in the 100 mg/kg QD treated animals, Compound C treatment was not efficacious and SDMA inhibition was less than 80%. This data suggests that SDMA levels need to be nearly completely inhibited (>90%) in order to see significant TGI in in vivo breast cancer xenograft models.
Overall, these data suggest PRMT5 inhibition as a potential therapeutic strategy in breast cancer, in particular TNBC subtype.

### Glioblastoma (GBM) Biology

PRMT5 protein is frequently overexpressed in glioblastoma tumors and high PRMT5 levels strongly correlate with both grade (grade IV) and poor survival in GBM patients (Yan F, et al. Cancer Res. 2014 Mar. 15; 74(6):1752-65). PRMT5 knockdown attenuates the growth and survival of GBM cell lines and significantly improves survival in an orthotopic Gli36 xenograft model (Yan F, et al. Cancer Res. 2014 Mar. 15; 74(6):1752-65). PRMT5 also plays an important role in normal mouse brain development through the regulation of growth and differentiation of neural progenitor cells (Bezzi M, et al. Genes Dev. 2013 Sep. 1; 27(17):1903-16).

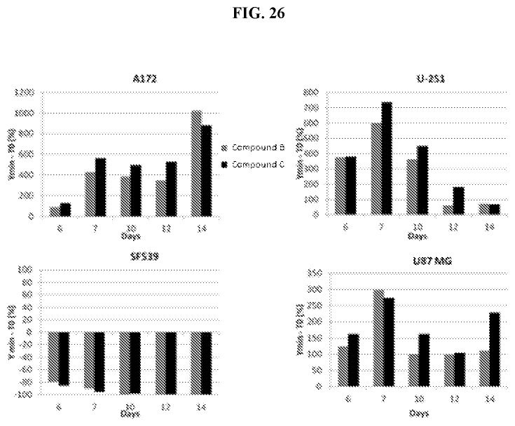

Glioblastoma cell line models were amongst the most sensitive tumor types in a soft agar colony formation assay (FIG. 10). In 2D, 6-day growth/death CTG assay, GBM cell lines had gIC₅₀ values in the 40-22000 nM range where the response was largely cytostatic, with the exception of the SF539 cell line (FIGS. 7 and 8). To understand the effects of PRMT5 inhibition on cell growth and survival upon longer exposure to a PRMT5 inhibitor, Compound C activity was tested in a 2D, 14-day growth/death CTG assay (FIG. 26). Overall, the nature of the cytostatic/cytotoxic response did not change upon longer exposure to the compound and the only cell line that underwent apoptosis in response to PRMT5 inhibition was SF539.

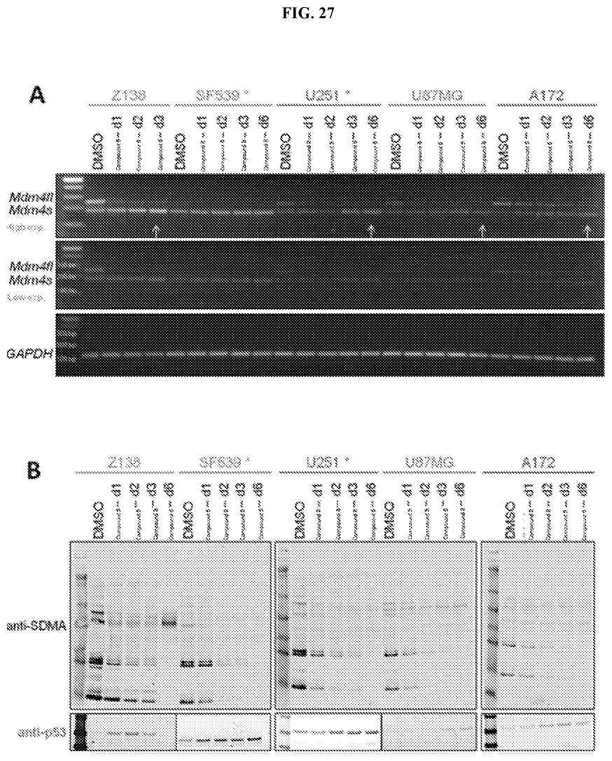

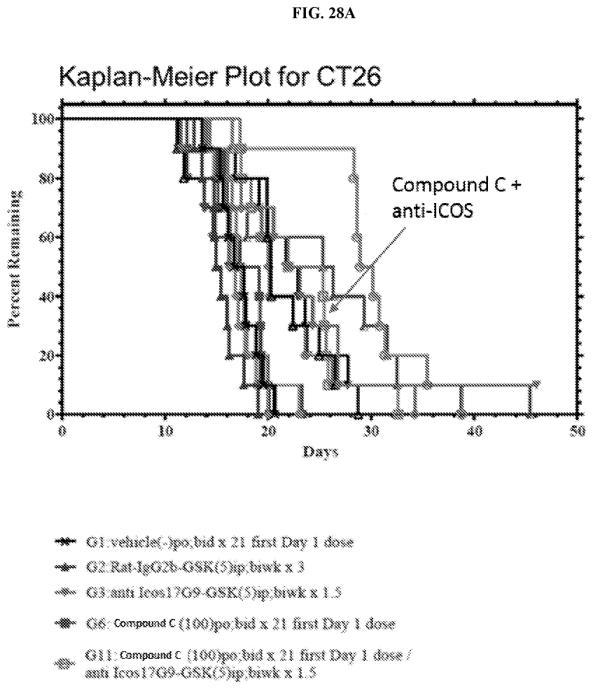

Next, effects on cellular methylation and the p53 pathway were evaluated in GBM cells treated with a PRMT5 inhibitor by measuring SDMA, p53 and p21 protein levels and MDM4 splicing (FIG. 27). PRMT5 inhibition resulted in the reduction of the SDMA signal in all cell lines tested (FIG. 27, panel B), irrespective of their sensitivity to PRMT5 inhibition. Alternative MDM4 splicing was detected in all cell lines but SF539 which are p53 mutant and have low basal expression of the long MDM4 isoform (FIG. 27, panel A). p53 levels increased in all cell lines, while the induction of p21 protein was observed only in cell lines that have wild-type p53 (Z138 (MCL), U87-MG and A172 (GBM)). These data suggest that PRMT5 inhibitors can activate the p53 pathway in GBM models, potentially through the inactivation of MDM4 activity, similar to the effects observed in lymphoma models. Importantly, GBM cell line sensitivity did not correlate with p53 mutational status, suggesting that additional mechanisms contribute to the growth inhibitory phenotypes induced by PRMT5 inhibition. Interestingly, PRMT5 inhibition resulted in a cytostatic response in wild-type p53 GBM cell lines. The role of p53 in the response of GBM cell lines to PRMT5 inhibition will be further tested in future studies. Additionally, the effects of PRMT5 inhibition on cell cycle and neural differentiation in GBM models are being explored.

### Glioblastoma Summary

PRMT5 protein is frequently overexpressed in glioblastoma tumors and high PRMT5 levels strongly correlate with high grade (grade IV) and poor survival in GBM patients.
Glioblastoma cell line models were amongst the most sensitive tumor types in a soft agar colony formation assay.
In 2D, 6- and 14-day growth/death CTG assays, GBM response to PRMT5 inhibition was largely cytostatic (3 out of 4 lines, 1 cell line had a cytotoxic response).
PRMT5 inhibition resulted in the reduction of the SDMA signal in all cell lines tested irrespective of their sensitivity to PRMT5 inhibition.

### Additional Sensitive Tumor Types

Cell line and patient-derived model screening data suggest that PRMT5 inhibitors attenuate cell growth and survival in a broad range of tumor types (FIGS. 7-10).

### Overall Biology Summary

Compound C inhibits symmetric arginine dimethylation on a variety of cellular proteins including spliceosome components, histones, transcription factors, and kinases. Therefore, PRMT5 inhibitors impact RNA homeostasis through a multitude of mechanisms including changes in transcription, splicing, and mRNA translation.
PRMT5 inhibition leads to gene expression and splicing changes ultimately resulting in the induction of p53. Compound C induces an isoform switch in the p53 ubiquitin ligase MDM4, stabilizes p53 protein, and induces p53 target gene expression signaling in mantle cell and diffuse large B-cell lymphoma as well as breast and glioma cancer cell lines (the only tumor types tested so far).
Compound C inhibits proliferation in a broad range of solid and heme tumor cell lines and induces cell death in a subset of mantle cell and diffuse large B-cell lymphoma, breast, bladder, and glioma cell lines. The most potent growth inhibition was observed in mantle cell and diffuse large B-cell lymphoma cell lines. Compound C efficacy was tested in xenograft models of mantle cell lymphoma and breast cancer, where it significantly inhibited tumor growth. These data provide strong rationale for the use of Compound C as a therapeutic strategy in mantle cell lymphoma, diffuse large B-cell lymphoma, breast and brain cancer.

### Example 2

### Combinations

Activity of ICOS Agonism in Combination with Inhibition of Type II PRMTs in Syngeneic Cancer Models

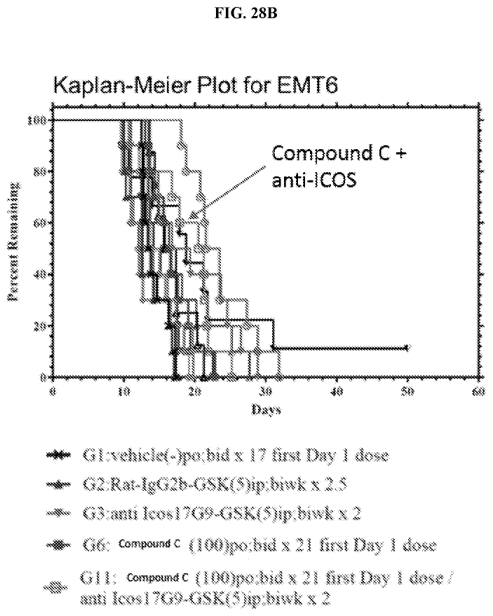

We explored whether the combination of type II PRMT inhibition by Compound C could increase the efficacy of an anti-ICOS antibody in immunocompetent tumor models. Compound C was dosed alone and in combination with an anti-ICOS agonist antibody (Icos17G9-GSK). FIG. 28A and FIG. 28B show the combination In both the CT26 and EMT6 tumor models, the combination provided survival benefit over either single agent (FIG. 28A, FIG. 28B).

The results described in Example 2 were obtained using the following materials and methods:

### Mice, Tumor Challenge and Treatment

7 week old female BALB/c mice (BALB/cAnNCrl, Charles River) were utilized for in-vivo studies in compliance with the USDA Laboratory Animal Welfare Act, in a fully accredited AAALAC facility (Charles River Laboratories). 3×10⁵ (CT26) or 5×10⁶ (EMT6) cells were inoculated sub-cutaneously into the right flank. Tumors were measured with calipers two times per week in two dimensions, and tumor volume was calculated using the formula: 0.5×Length×Width². Mice (n=10/treatment group) were randomized when the tumors reached 100 to 150 mm³ and received saline (once daily, oral administration), 100 mg/kg Compound C (twice daily, oral administration), 5 mg/kg anti-ICOS (17G9; twice weekly via intraperitoneal injection), or the combination of Compound C and anti-ICOS. For all studies, Compound C was administered for 3 weeks; CT26 and EMT6 models received 3 or 4 doses of anti-ICOS antibody, respectively. Tumor measurement of greater than 2,000 mm³ for an individual mouse and/or development of open ulcerations resulted in mice being removed from study.

1. A method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding portion thereof.

2. The method of claim 1, wherein the Type II PRMT inhibitor is a protein arginine methyltransferase 5 (PRMT5) inhibitor or a protein arginine methyltransferase 9 (PRMT9) inhibitor.

3. The method of claim 1, wherein the Type II PRMT inhibitor is a compound of Formula (III): 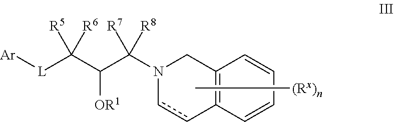
or a pharmaceutically acceptable salt thereof,
wherein [symbol]
represents a single or double bond;
R¹ is hydrogen, R^{z}, or —C(O)R^{z}, wherein R^{z} is optionally substituted C₁₋₆ alkyl;
L is —N(R)C(O)—, —C(O)N(R)—, —N(R)C(O)N(R)—, —N(R)C(O)O—, or —OC(O)N(R)—;
each R is independently hydrogen or optionally substituted C₁₋₆ aliphatic;
Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits;
each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NORA)R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A} or —SO₂N(R^{B})₂;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;
R⁵, R⁶, R⁷, and R⁸ are independently hydrogen, halo, or optionally substituted aliphatic;
each R^{X} is independently selected from the group consisting of halo, —CN, optionally substituted aliphatic, —OR′, and —N(R^{ff})₂;
R′ is hydrogen or optionally substituted aliphatic;
each R″ is independently hydrogen or optionally substituted aliphatic, or two R″ are taken together with their intervening atoms to form a heterocyclic ring; and
n is 0, 1, 2, 3, 4, 5, 6, 7, 8, 9, or 10, as valency permits.

4. The method of claim 1, wherein the Type II PRMT inhibitor is a compound of Formula (X): 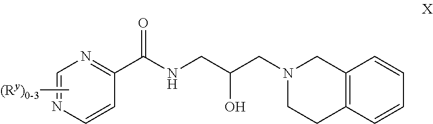
or a pharmaceutically acceptable salt thereof.

5. The method of claim 1, wherein the Type II PRMT inhibitor is Compound C: 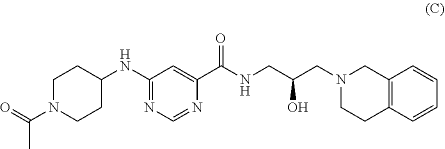 [SMILES: CC(=O)N1CCC(Nc2cc(C(=O)NC[C@H](O)CN3CCc4ccccc4C3)ncn2)CC1]
or a pharmaceutically acceptable salt thereof.

6. The method of claim 1, wherein the ICOS binding protein is an anti-ICOS antibody or antigen binding fragment thereof.

7. The method of claim 1, wherein the ICOS binding protein is an ICOS agonist.

8. The method of claim 1, wherein the ICOS binding protein or antigen binding portion thereof comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

9. The method of claim 1, wherein the ICOS binding protein or antigen binding portion thereof comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS.

10. A method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding fragment thereof, wherein the Type II PRMT inhibitor is Compound C: 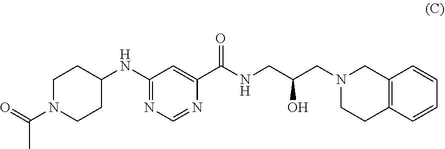 [SMILES: CC(=O)N1CCC(Nc2cc(C(=O)NC[C@H](O)CN3CCc4ccccc4C3)ncn2)CC1]
or a pharmaceutically acceptable salt thereof, and the ICOS binding fragment or antigen binding fragment thereof comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

11. A method of treating cancer in a human in need thereof, the method comprising administering to the human a therapeutically effective amount of a Type II protein arginine methyltransferase (Type II PRMT) inhibitor and administering to the human a therapeutically effective amount of an ICOS binding protein or antigen binding fragment thereof, wherein the Type II PRMT inhibitor is Compound C: 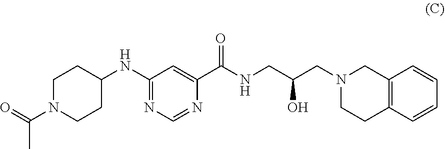 [SMILES: CC(=O)N1CCC(Nc2cc(C(=O)NC[C@H](O)CN3CCc4ccccc4C3)ncn2)CC1]
or a pharmaceutically acceptable salt thereof, and the ICOS binding protein or antigen binding portion thereof comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS.

12-22. (canceled)

23. The method of claim 1, wherein the Type I IPRMT inhibitor or the ICOS binding protein or antigen binding fragment thereof is administered to the patient in a route selected from: simultaneously, sequentially, in any order, systemically, orally, intravenously, and intratumorally.

24. The method of claim 1 wherein the Type II PRMT inhibitor is administered orally.

25. The method of claim 1 wherein the ICOS binding protein or antigen binding fragment thereof is administered intravenously.

26. The method of claim 1 wherein the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, prostate cancer, and lymphoma.

27-28. (canceled)


SEQUENCE LISTING (18 sequences)

SEQ ID NO: 1 - protein, 5 aa, Artificial Sequence
  Amino acid sequence identified using molecular biology techniques
DYAMH

SEQ ID NO: 2 - protein, 17 aa, Artificial Sequence
  Amino acid sequence identified using molecular biology techniques
LISIYSDHTNYNQKFQG

SEQ ID NO: 3 - protein, 12 aa, Artificial Sequence
  Amino acid sequence identified using molecular biology techniques
NNYGNYGWYFDV

SEQ ID NO: 4 - protein, 10 aa, Artificial Sequence
  Amino acid sequence identified using molecular biology techniques
SASSSVSYMH

SEQ ID NO: 5 - protein, 7 aa, Artificial Sequence
  Amino acid sequence identified using molecular biology techniques
DTSKLAS

SEQ ID NO: 6 - protein, 9 aa, Artificial Sequence
  Amino acid sequence identified using molecular biology techniques
FQGSGYPYT

SEQ ID NO: 7 - protein, 121 aa, Artificial Sequence
  Amino acid sequence identified using molecular biology techniques
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  Amino acid sequence identified using molecular biology techniques
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 168 aa, Artificial Sequence
  Amino acid sequence identified using molecular biology techniques
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 10 - protein, 199 aa, Artificial Sequence
  Amino acid sequence identified using molecular biology techniques
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 11 - protein, 116 aa, Artificial Sequence
  Amino acid sequence identified using molecular biology techniques
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 12 - protein, 111 aa, Artificial Sequence
  Amino acid sequence identified using molecular biology techniques
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 13 - protein, 10 aa, Artificial Sequence
  Amino acid sequence identified using molecular biology techniques
GFTFSDYWMD

SEQ ID NO: 14 - protein, 17 aa, Artificial Sequence
  Amino acid sequence identified using molecular biology techniques
NIDEDGSITEYSPFVKG

SEQ ID NO: 15 - protein, 8 aa, Artificial Sequence
  Amino acid sequence identified using molecular biology techniques
WGRFGFDS

SEQ ID NO: 16 - protein, 15 aa, Artificial Sequence
  Amino acid sequence identified using molecular biology techniques
KSSQSLLSGSFNYLT

SEQ ID NO: 17 - protein, 7 aa, Artificial Sequence
  Amino acid sequence identified using molecular biology techniques
YASTRHT

SEQ ID NO: 18 - protein, 9 aa, Artificial Sequence
  Amino acid sequence identified using molecular biology techniques
HHHYNAPPT